(19) United States
(12) Patent Application Publication
(10) Pub. No.: US 2006/0252682 A1
(43) Pub. Date: Nov. 9, 2006

(54) Liquid growth hormone formulation and process of preparation thereof

(75) Inventors: Piergiorgio Donati, Morges (CH); Fabrizio Samaritani, Rome (IT)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Ares Trading S.A., Aubonne (CH)

(21) Appl. No.: 10/549,763

(22) PCT Filed: Mar. 11, 2004
(86) PCT No.: PCT/EP04/50286

§ 371 Date: May 11, 2006

(30) Foreign Application Priority Data
Mar. 18, 2003 (EP) 03100678.6

(51) Int. Cl. A61K 38/27

(52) U.S. Cl. 514/12

(57) ABSTRACT

The invention relates to a liquid formulation comprising a growth hormone or a, substance, which stimulates release or potentiates the activity of endogenous hGH; a polyethylene-polypropylene glycol; a citrate buffer and a stabilizer, and to a process of preparation thereof.

### FIELD OF THE INVENTION

This invention relates to liquid growth hormone (GH) formulations, and in particular to liquid formulations of human growth hormone (hGH) which significantly improve solubility of growth hormone and remain substantially free from particulate matter over a prolonged period of time. The present invention further relates to a process for the preparation of such liquid GH formulations, and to a form of presentation thereof.

### BACKGROUND OF THE INVENTION

Human growth hormone (hGH), also known as somatropin (INN) or somatotropin, is a protein hormone produced and secreted by the somatotropic cells of the anterior pituitary. Human growth hormone plays a key role in somatic growth in childhood and in metabolism in adulthood through its effects on the metabolism of proteins, carbohydrates and lipids.

Human growth hormone is a single polypeptide chain of 191 amino acids (Bewly et al, 1972) having two disulfide bonds, one between Cys-53 and Cys-165, forming a large loop in the molecule, and the other between Cys-182 and Cys-189, forming a small loop near the C-terminus. The DNA sequence that confirmed the amino acid sequence was reported by Martial et al (1979). Purified hGH is a white amorphous powder in its lyophilized form. It is readily soluble (concentrations>10 mg/L) in aqueous buffers at pH in a range of 6.5 to 8.5.

In solution, hGH exists predominantly as a monomer, with a small fraction as dimers and higher molecular weight oligomers. Under certain conditions, hGH can be induced to form larger amounts of dimers, trimers and higher oligomers.

Several derivatives of hGH are known, including naturally-occurring derivatives, variants and metabolic products, degradation products primarily of biosynthetic hGH and engineered derivatives of hGH produced by genetic methods. One example of a naturally-occurring derivative of hGH is GH-V, a variant of growth hormone found in the placenta. Other members of the gene locus are described in Chen et al (1989).

Methionyl hGH was the first form of hGH to be produced through recombinant DNA technology. This compound is actually a derivative of hGH having one additional methionine residue at its N-terminus (Goeddel et al, 1979).

A naturally-occurring variant of hGH called 20-K-hGH has been reported to occur in the pituitary as well as in the bloodstream (Lewis et al, 1978; Lewis et al, 1980). This compound, which lacks the 15 amino acid residues from Glu-32 to Gln-46, arises from an alternative splicing of the messenger ribonucleic acid (DeNoto et al, 1981). This compound shares many, but not all of the biological properties of hGH.

20-K-hGH is made in the pituitary and secreted into the blood. It makes up about 5% of growth hormone output of adults, and about 20% of growth hormone output of children. It has the same growth promoting activity as 22 kD growth hormone, and has been reported to have equal to or greater the amount of lipolytic activity as the 22 kD form. It binds to growth hormone receptors with equal affinity as the 22 kD growth hormone, and has one tenth the lactogenic (prolactin-like) bioactivity as the 22 kD hormone. Unlike 22 kD, the 20-k-hGH has weak anti-insulin activity.

A number of derivatives of hGH arise from proteolytic modifications of the molecule. The primary pathway for the metabolism of hGH involves proteolysis. The region of hGH around residues 130-150 is extremely susceptible to proteolysis, and several derivatives of hGH having nicks or deletions in this region have been described (Thorlacius-Ussing, 1987). This region is in the large loop of hGH, and cleavage of a peptide bond there results in the generation of two chains that are connected through the disulfide bond at Cys-53 and Cys-165. Many of these two-chain forms are reported to have increased biological activity (Singh et al, 1974). Many derivatives of human growth hormone have been generated artificially through the use of enzymes. The enzymes trypsin and subtilisin, as well as others, have been used to modify hGH at various points throughout the molecule (Lewis et al, 1977; Graff et al, 1982). One such derivative, called two-chain anabolic protein (2-CAP), was formed through the controlled proteolysis of hGH using trypsin (Becker et al, 1989). 2-CAP was found to have biological properties very distinct from those of the intact hGH molecule, in that the growth-promoting activity of hGH was largely retained and most of the effects on carbohydrate metabolism were abolished.

Asparagine and glutamine residues in proteins are susceptible to deamidation reactions under appropriate conditions. Pituitary hGH has been shown to undergo this type of reaction, resulting in conversion of Asn-152 to aspartic acid and also, to a lesser extent, conversion of Gln-137 to glutamic acid (Lewis et al, 1981). Deamidated hGH has been shown to have an altered susceptibility to proteolysis with the enzyme subtilisin, suggesting that deamidation may have physiological significance in directing proteolytic cleavage of hGH. Biosynthetic hGH is known to degrade under certain storage conditions, resulting in deamidation at a different asparagine (Asn-149). This is the primary site of deamidation, but deamidation at Asn-152 is also seen (Becker et al, 1988). Deamidation at Gln-137 has not been reported in biosynthetic hGH.

Methionine residues in proteins are susceptible to oxidation, primarily to the sulfoxide. Both pituitary-derived and biosynthetic hGH undergo sulfoxidations at Met-14 and Met-125 (Becker et al, 1988). Oxidation at Met-170 has also been reported in pituitary but not biosynthetic hGH. Both desamide hGH and Met-14 sulfoxide hGH have been found to exhibit full biological activity (Becker et al, 1988).

Truncated forms of hGH have been produced, either through the actions of enzymes or by genetic methods. 2-CAP, generated by the controlled actions of trypsin, has the first eight residues at the N-terminus of hGH removed. Other truncated versions of hGH have been produced by modifying the gene prior to expression in a suitable host. The first 13 residues have been removed to yield a derivative having distinctive biological properties (Gertler et al, 1986) in which the polypeptide chain is not cleaved.

Although human growth hormone was originally obtained from pituitary glands of cadavers, these preparations were not electrophoretically homogeneous, and antibodies appeared in the serum of patients treated with preparations of the order of 50% purity, the immunogenicity being attributed to inactive components. Recombinant DNA technology permitted production of an unlimited supply of hGH in a number of different systems. Purification of hGH from the culture medium is facilitated by the presence of only low amounts of contaminating proteins. In fact, it has been shown that hGH can be purified on a laboratory scale by a single purification step on a reversed-phase HPLC column (Hsiung et al (1989).

Recombinant human growth hormone, rhGH, is produced by Serono International S. A. as SEROSTIM®, which product has been given accelerated FDA approval for treating weight loss and wasting in AIDS patients. SAIZEN® is recombinant human growth hormone indicated for GH deficiency in children, for Turner syndrome in girls, as well as chronic renal failure in children. PROTROPIN®, produced by Genentech, Inc. (South San Francisco, Calif.), differs slightly in structure from natural sequence hGH, having an additional methionine residue at the N-terminus. Recombinant hGH is generally marketed as vials containing hGH plus additional excipients, e.g., glycine and mannitol, in a lyophilized form. A companion diluent vial is provided, allowing the patient to reconstitute the product to the desired concentration prior to administration of the dose. Recombinant hGH can also be marketed in other well-known manners, such as pre-filled syringes.

In general, no significant differences have been observed in the pharmacokinetics or biological activities of recombinant natural sequence hGH, recombinant N-methionyl-hGH, or pituitary-derived material in humans (Moore et al, 1988; Jorgensson et al, 1988).

In order for hGH to be available commercially as a therapeutic, stable formulations must be prepared. Such formulations must be capable of maintaining activity for appropriate storage times and be acceptable for administration by patients.

Human GH has been formulated in a variety of ways. By way of example, U.S. Pat. No. 5,096,885 discloses a stable pharmaceutically acceptable formulation of hGH comprising, in addition to the hGH, glycine, mannitol, a buffer and optionally a non-ionic surfactant, the molar ratio of hGH:glycine being 1:50.

WO 93/19776 discloses injectable formulations of GH comprising citrate as buffer substance and optionally growth factors such as insulin-like growth factors or epidermal growth factor, amino acids such as glycine or alanine, mannitol or other sugar alcohols, glycerol and/or a preservative such as benzyl alcohol.

WO 94/101398 discloses a GH formulation containing hGH, a buffer, a non-ionic surfactant and, optionally, mannitol, a neutral salt and/or a preservative.

EP-0131864 describes an aqueous solution of proteins with molecular weight above 8500 daltons, which have been protected from adsorption at interfaces, against denaturing and against precipitation of the protein by addition of a linear polyoxyalkylene chain-containing surface-active substance as a stabilising agent.

EP-0211601 discloses a growth promoting formulation comprising an aqueous mixture of growth promoting hormone and a block copolymer containing polyoxyethylene-polyoxypropylene units and having an average molecular weight of about 1,100 to about 40,000 which maintains the fluidity of the growth promoting hormone and its biological activity upon administration.

WO 97/29767 discloses a liquid formulation comprising a growth hormone, trisodium citrate dihydrate, sodium chloride, sodium hydroxide, benzyl alcohol, Pluronic F-68, said formulation having a pH of 5.6.

U.S. Pat. No. 5,567,677 discloses liquid formulations comprising human growth hormone, sodium citrate, sodium phosphate, glycine, mannitol, optionally benzyl alcohol.

Pharmaceutical preparations of hGH tend to be unstable, particularly in solution. Chemically degraded species such as deamidated or sulfoxylated forms of hGH occur, and dimeric or higher molecular weight aggregated species may result from physical instability (Becker et al (1988); Becker et al., 1987; Pearlman and Nguyen (1989)).

As a consequence of the instability of hGH in solution, pharmaceutical formulations of hGH are generally in lyophilised form, which must then be reconstituted prior to use. Reconstitution is usually carried out by the addition of a pharmaceutically acceptable diluent such as sterile water for injection, sterile physiological saline or an appropriate sterile physiologically acceptable diluent.

Reconstituted solutions of hGH are preferably stored at 4° C. to minimise chemical and physical degradation reactions, however some degradation will occur during such storage which can be for a period of up to 14 days.

A pharmaceutical formulation of hGH provided in a liquid form, particularly one that maintaines stability of hGH without formation of precipitation or aggregation or any other particulate matter over a prolonged period of time, would be particularly advantageous.

Therefore, it is an object of the present invention to provide liquid formulations of growth hormone that do not result in the formation of undesirable particulate matter and that has a prolonged storage time.

### SUMMARY OF THE INVENTION

The solubility of growth hormone can be significantly increased by addition of a polyethylene-polypropylene glycol to the liquid formulation. The liquid growth hormone formulation may be stored at both room temperature and +5° C. if citrate buffer is used.

Therefore, a first aspect of the invention therefore relates to a liquid formulation comprising

a growth hormone or a substance, which stimulates release or potentiates the activity of endogenous hGH;

a polyethylene-polypropylene glycol;

a citrate buffer; and

a stabilizer.

A second aspect of the invention relates to a process for production of the liquid formulation in accordance with the present invention.

In a third aspect, the invention relates to the use of a formulation according to the invention for mono-dose or multi-dose administration of a growth hormone.

A fourth aspect of the invention relates to a form of presentation of the liquid formulation according to the invention.

### BRIEF DESCRIPTION OF THE DRAWINGS

FIG. 1: RP-HPLC results after 4 weeks storage at +25±2° C. of r-hGH liquid formulations containing Mannitol or Sucrose or no excipient;

FIG. 2: RP-HPLC results after 4 weeks storage at +25±2° C. of r-hGH liquid formulations containing tensioactive at various concentrations and no tensioactive;

FIG. 3: Regression lines of lab scale formulations tested by RP-HPLC for related proteins up to 6 months storage at +25±2° C.;

FIG. 4: Regression lines of lab scale candidate formulations #3, #4 and #5 tested by RP-HPLC for related proteins up to 6 months storage at +25±2° C.;

FIG. 5: pH of lab scale formulations up to 6 months storage at 25±2° C.;

FIG. 6: pH of lab scale formulations up to 12 months storage at 5±3°;

FIG. 7: stability of hGH in multi-dose formulations 6 (squares), 7 (circles) and 8 (diamonds) at +5° C.

FIG. 8: stability of hGH in multi-dose formulations 6 (squares), 7 (triangles) and 8 (circles) at +5° C.

FIG. 9: stability of hGH in different multi-dose formulations at +25° C. in months.

FIG. 10: stability of hGH in different multi-dose formulations at +5° C. in months.

FIG. 11 pH of the multi-dose formulations comprising acetate (squares and diamonds), citric acid (triangles) or citrate (half squares) at +25° C. over 6 months.

### DETAILED DESCRIPTION OF THE INVENTION

In the frame of the present invention, it has been found that a specific group of surfactants significantly improves solubility of growth hormone and thus renders it possible to obtain solutions substantially free from particulate matter.

It has further been found that improved stability of growth hormone in liquid formulation over long periods of time can be obtained with citrate as a buffer.

Therefore, the invention relates to a liquid formulation comprising

a growth hormone, or a substance, which stimulates release or potentiates the activity of endogenous hGH;

a polyethylene-polypropylene glycol;

a citrate buffer; and

a stabilizer.

Growth hormone that may be formulated in accordance with the present invention may be derived from any species, such as bovine, porcine, canine or feline, depending on the intended use of the formulation. A substance, which stimulates release or potentiates the activity of endogenous hGH, is e.g. growth hormone releasing hormone.

Preferably, the following substances may be formulated in accordance with the present invention:
a) human growth hormone;
b) a fragment of (a) which has agonistic activity on the hGH receptor;
c) a variant of (a) or (b) which has at least 70% sequence identity with (a) or (b) and which has agonistic activity on the hGH receptor;
d) a variant of (a) or (b) which is encoded by a DNA sequence which hybridizes to the complement of the native DNA sequence encoding (a) or (b) under moderately stringent conditions and which has agonistic activity on the hGH receptor; or
e) a salt or functional derivative of (a), (b), (c) or (d) which has agonistic activity on the hGH receptor.

A formulation comprising human growth hormone is preferred in accordance with the present invention.

The term “human growth hormone”, or “hGH”, as used in the present invention, is intended to include the naturally-occurring and synthetic derivatives, as noted above, including, without limitation, both the 20 kD and the 22 kD human growth hormone, GH-V, and other members of the growth hormone gene locus, as described in detail in the “Background of the invention”.

The hGH may be naturally-occurring human growth hormone, or it may preferably be recombinant hGH. Recombinant GH may be expressed in any suitable host, either a prokaryotic, or a eukaryotic host. E. coli is a host particularly suitable for expression of hGH, for instance. Yeast, insect, or mammalian cells are further suitable for expression of recombinant growth hormone. Preferably, the hGH is expressed in human or animal cells, e.g. in Chinese Hamster Ovary (CHO) cells.

The term “hGH” or “growth hormone”, as used herein, also includes functional derivatives, fragments, variants, analogs, or salts which retain the biological activity of growth hormone, i.e., which act as agonists to the growth hormone receptor. In other words, they are capable of binding to the growth hormone receptor to initiate the signaling activity of the receptor.

The term “functional derivatives”, or “chemical derivatives”, as used herein covers derivatives which may be prepared from the functional groups which occur as side chains on the residues of the N- or C-terminal groups, by means known in the art, and are included in the invention as long as they remain pharmaceutically acceptable, and do not destroy the biological activity of hGH as described herein, i.e., the ability to bind the hGH receptor and initiate receptor signaling, and do not confer toxic properties on compositions containing it. Derivatives may have chemical moieties, such as carbohydrate or phosphate residues, provided such a derivative retains the biological activity of hGH and remains pharmaceutically acceptable.

For example, derivatives may include aliphatic esters of the carboxyl groups, amids of the carboxyl groups by reaction with ammonia or with primary or secondary amines, N-acyl derivatives or free amino groups of the amino acid residues formed with acyl moieties (e.g., alkanoyl or carbocyclic aroyl groups) or O-acyl derivatives of free hydroxyl group (e.g., that of seryl or threonyl residues) formed with acyl moieties. Such derivatives may also include for example, polyethylene glycol side-chains, which may mask antigenic sites and extend the residence of the molecule in body fluids.

A growth hormone that has been derivatized or combined with a complexing agent may be long lasting. Therefore, a preferred embodiment of the invention relates to PEGylated versions of human growth hormone. Growth hormones genetically engineered to exhibit long lasting activity in the body, are also examples for hGH derivatives within the scope of the present invention.

hGH that is acetylated at the N-terminus has been isolated and identified (Lewis et al, 1979). It is not clear if acylation serves a regulatory role or is simply an artifact of the purification. However, it is expected that this molecule exhibits GH activity in a similar fashion to other hGH derivatives. Therefore, in a preferred embodiment, the invention relates to human growth hormone which is acetlyated at its N-terminus.

Preferably, the formulation according to the invention comprises a dimer of human growth hormone selected from the group consisting of a disulfide dimer connected through interchain disulfide bonds, a covalent irreversible non-disulfide dimer, a non-covalent dimer, and mixtures thereof.

The term “salts” herein refers to both salts of carboxyl groups and to acid addition salts of amino groups of the hGH molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of hGH relevant to the present invention, i.e., the ability to bind to the hGH receptor and initiate receptor signaling.

In a further preferred embodiment, the invention relates to fragment of human growth hormone.

A “fragment” of the growth hormone according to the present invention refers to any subset of the molecule, that is, a shorter peptide, which retains the desired biological activity. Fragments may readily be prepared by removing amino acids from either end of the hGH molecule and testing the resultant for its properties as an hGH receptor agonist. Proteases for removing one amino acid at a time from either the N-terminal or the C-terminal of a polypeptide are known, and so determining fragments which retain the desired biological activity involves only routine experimentation.

Preferably, hGH fragments in accordance with the present invention may have internal deletions, as long as the deletion does not affect the biological activity of hGH, i.e. binding to and initiating signaling through the hGH receptor. A fragment that is preferred according to the invention lacks 15 amino acids from Glutamic acid (Glu) 32 to Glutamic acid 46.

hGH fragments may further be truncated at the C- or N-terminus. Truncated hGH lacking the first eight N-terminal residues or the first 13 N-terminal residues of human growth hormone are also preferred in accordance with the present invention.

A short C-terminal hGH fragment had been described to retain a biological activity of hGH, see U.S. Pat. No. 5,869,452. Therefore, the use of a C-terminal fragment of hGH is preferred according to the invention. Fragment hGH177-191, comprising at least amino acid residues 177 to 191 of hGH (LRIVQCRSVEGSCGF) is particularly preferred in accordance with the present invention. Further preferred are derivatives of this peptide, such as the peptide variants described in U.S. Pat. No. 6,335,319 or WO99/12969, e.g. cyclic peptides.

Additionally, the polypeptide, which has such hGH receptor agonist activity, be it hGH, an analog or variant, salt, functional derivative or fragment thereof, can also contain additional amino acid residues flanking the hGH polypeptide. As long as the resultant molecule retains the hGH receptor agonist ability of the core polypeptide, one can determine whether any such flanking residues affect the basic and novel characteristics of the core peptide, i.e., its receptor agonist characteristics, by routine experimentation.

An example for such a GH variant, which is preferred in accordance with the present invention, is methionyl human growth hormone (Met-hGH), which has an additional methionine residue at the N-terminus of human growth hormone.

Variants of hGH, which are preferred according to the invention, comprise methionyl hGH, which is a human growth hormone having an additional methionine residue at its N-terminus. A further preferred variant is a human growth hormone lacking 15 amino acid residues from Glu32 to Glu46.

A “variant” of the human growth hormone according to the present invention refers to a molecule, which is substantially similar to either the entire protein or a fragment thereof. A variant may also be called a “mutein”. A variant may e.g. be an isoform of hGH, such as a variant generated by alternative splicing. Variant (poly)peptides may also be conveniently prepared by direct chemical synthesis of the variant peptide, using methods well known in the art. Of course, a variant human growth hormone would have at least similar hGH receptor binding and signal initiating activity as hGH and which would, therefore, be expected to have similar activity to hGH.

Amino acid sequence variants of the human growth hormone can be prepared by mutations in the DNAs, which encode the synthesized human growth hormone derivatives. Such variants include, for example, deletions from, or insertions or substitutions of, residues within the amino acid sequence. Any combination of deletion, insertion, and substitution may also be made to arrive at the final construct, provided that the final construct possesses the desired activity. Obviously, the mutations that will be made in the DNA encoding the variant peptide must not alter the reading frame.

At the genetic level, these variants may be prepared by site-directed mutagenesis (as exemplified by Adelman et al, 1983) of nucleotides in the DNA encoding the peptide molecule, thereby producing DNA encoding the variant, and thereafter expressing the DNA in recombinant cell culture. The variants typically exhibit at least the same qualitative biological activity as the non-variant peptide.

An “analog” of human growth hormone according to the present invention refers to a non-natural molecule, which is substantially similar to either the entire molecule or to an active fragment thereof. An analog of human growth hormone useful in the present invention would exhibit GH activity.

The types of substitutions which may be made in the human growth hormone according to the present invention may be based on analysis of the frequencies of amino acid changes between a homologous protein of different species. Based upon such analysis, conservative substitutions may be defined herein as exchanges within one of the following five groups:
I. Small, aliphatic, nonpolar or slightly polar residues:
   Ala, Ser, Thr, Pro, Gly
II. Polar, negatively-charged residues and their amides:
   Asp, Asn, Glu, Gln
III. Polar, positively-charged residues:
   His, Arg, Lys
IV. Large, aliphatic non-polar residues:
   Met, Leu, Ile, Val, Cys
V. Large aromatic residues:
   Phe, Try, Trp

Within the foregoing groups, the following substitutions are considered to be “highly conservative”:
Asp/Glu
His/Arg/Lys
Phe/Tyr/Trp
Met/Leu/Ile/Val

Semi-conservative substitutions are defined to be exchanges between two of groups (I)-(IV) above which are limited to supergroup (A), comprising (I), (II), and (III) above, or to supergroup (B), comprising (IV) and (V) above. Substitutions are not limited to the genetically encoded or even the naturally-occurring amino acids. When the epitope is prepared by peptide synthesis, the desired amino acid may be used directly. Alternatively, a genetically encoded amino acid may be modified by reacting it with an organic derivatizing agent that is capable of reacting with selected side chains or terminal residues.

Cysteinyl residues most commonly are reacted with alpha-haloacetates (and corresponding amines), such as chloroacetic acid or chloroacetamide, to give carboxylmethyl or carboxyamidomethyl derivatives. Cysteinyl residues also are derivatized by reaction with bromotrifluoroacetone, alpha-bromo-beta-(5-imidazoyl)propionic acid, chloroacetyl phosphate, N-alkylmaleimides, 3-nitro-2-pyridyl disulfide, methyl-2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Parabromophenacyl bromide is also useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino acid-containing residues include imidoesters such as methyl picolinimidate; pyridoxal phosphate; pyridoxal; chloroborohydride; trinitrobenzenesulfonic acid; O-methyliosurea; 2,4-pentanedione; and transaminase-catalyzed reaction with glyoxylate.

Arginyl residues are modified by reaction with one or several conventional reagents, among them phenylglyoxal; 2,3-butan edione; and ninhydrin. Derivatization of arginine residues requires that the reaction be performed in alkaline conditions because of the high pKa of the guanidine functional group. Furthermore, these reagents may react with the groups of lysine, as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidazole and tetranitromethane are used to form O-acetyl tyrosyl species and ε-nitro derivatives, respectively.

Carboxyl side groups (aspartyl or glutamyl) are selectively modified by reaction with carbodiimides (R′N—C—N—R′) such as 1-cyclohexyl-3-[2-morpholinyl-(4-ethyl)]carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Examples of production of amino add substitutions in proteins which can be used for obtaining analogs of the hGH for use in the present invention include any known method steps, such as presented in U.S. Pat. RE 33,653; U.S. Pat. Nos. 4,959,314; 4,588,585 and 4,737,462, to Mark et al; U.S. Pat. No. 5,116,943 to Koths et al; U.S. Pat. No. 4,965,195 to Namen et al; and U.S. Pat. No. 5,017,691 to Lee, et al, and lysine substituted proteins presented in U.S. Pat. No. 4,904,584 (Shaw et al). Further growth hormone variants have been described e.g. in U.S. Pat. No. 6,143,523 (Cunningham et al.).

Among the substances which bind to and initiate signaling of the human growth hormone receptor which may be used in accordance with the present invention are all of those growth hormone analogs and mimetics already known in the literature, such as, for example, those disclosed in U.S. Pat. Nos. 5,851,992; 5,849,704; 5,849,700; 5,849,535; 5,843,453; 5,834,598; 5,688,666; 5,654,010; 5,635,604; 5,633,352; 5,597,709; and 5,534,617.

Preferably, the hGH variant or analog will have a core sequence, which is the same as that of the native sequence or biologically active fragment thereof, which has an amino acid sequence having at least 70% identity to the native amino acid sequence and retains the biological activity thereof. More preferably, such a sequence has at least 80% identity, at least 90% identity, or most preferably at least 95% identity to the native sequence.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9.1 (Devereux J et al., 1984), for example the programs BESTFIT and GAP, may be used to determine the % identity between two polynucleotides and the % identity and the % homology between two polypeptide sequences. BESTFIT uses the “local homology” algorithm of Smith and Waterman (1981) and finds the best single region of similarity between two sequences. Other programs for determining identity and/or similarity between sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson W R, 1990; Pearson 1988).

Preferred changes for variants or muteins in accordance with the present invention are what are known as “conservative” substitutions. Conservative amino acid substitutions of growth hormone polypeptides or proteins, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the purview of the present invention.

Analogs or variants in accordance with the present invention may also be determined in accordance with the following procedure. The DNA of the native sequence is known to the prior art and is found in the literature (Martial et al, 1979). Polypeptides encoded by any nucleic acid, such as DNA or RNA, which hybridizes to the complement of the native DNA or RNA under highly stringent or moderately stringent conditions, as long as that polypeptide maintains the biological activity of the native sequence, are also considered to be within the scope of the present invention.

Stringency conditions are a function of the temperature used in the hybridization experiment, the molarity of the monovalent cations and the percentage of formamide in the hybridization solution. To determine the degree of stringency involved with any given set of conditions, one first uses the equation of Meinkoth et al. (1984) for determining the stability of hybrids of 100% identity expressed as melting temperature Tm of the DNA-DNA hybrid:
Tₘ=81.5° C.+16.6(_{Log}M)+0.41(% GC)−0.61(% form)−500/L
where M is the molarity of monovalent cations, % GC is the percentage of G and C nucleotides in the DNA, % form is the percentage of formamide in the hybridization solution, and L is the length of the hybrid in base pairs. For each 1° C. that the Tm is reduced from that calculated for a 100% identity hybrid, the amount of mismatch permitted is increased by about 1%. Thus, if the Tm used for any given hybridization experiment at the specified salt and formamide concentrations is 10° C. below the Tm calculated for a 100% hybrid according to equation of Meinkoth, hybridization will occur even if there is up to about 10% mismatch.

As used herein, highly stringent conditions are those which are tolerant of up to about 15% sequence divergence, while moderately stringent conditions are those which are tolerant of up to about 20% sequence divergence. Without limitation, examples of highly stringent (12-15° C. below the calculated Tm of the hybrid) and moderately (15-20° C. below the calculated Tm of the hybrid) conditions use a wash solution of 2×SSC (standard saline citrate) and 0.5% SDS at the appropriate temperature below the calculated Tm of the hybrid. The ultimate stringency of the conditions is primarily due to the washing conditions, particularly if the hybridization conditions used are those, which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6×SSPE), 5× Denhardt's reagent, 0.5% SDS, 100 μg/ml denatured, fragmented salmon sperm DNA at a temperature approximately 200 to 25° C. below the Tm. If mixed probes are used, it is preferable to use tetramethyl ammonium chloride (TMAC) instead of SSC (Ausubel, 1987-1998).

While the present invention provides recombinant methods for making the human growth hormone derivatives, these derivatives may also be made by conventional protein synthesis methods which are well known to those skilled in the art.

The formulation of the invention comprises polyethylene-polypropylene glycol. This polymer is a nonionic surfactant. A surfactant may herein also be called “tensioactive” or “tensioactive agent”. In yet a further preferred embodiment, the formulation comprises the polyethylene-polypropylene glycol in a concentration ranging from 0.5 to 5 mg/ml or 1 to 2 mg/ml or 1.5 mg/ml.

In a preferred formulation, the surfactant is a pluronic polyol, such as for instance F68. Pluronic F68 is highly preferred in accordance with the present invention.

By formulating GH with the surfactant Pluronic® F68 (BASF, also known as Poloxamer 188) a stable formulation was obtained that avoids the problem of precipitation, aggregation or generation of particulate matter of any kind.

Pluronic F68 is a block copolymer of ethylene oxide (EO) and propylene oxide (PO). The propylene oxide block (PO) is sandwiched between two ethylene oxide (EO) blocks.

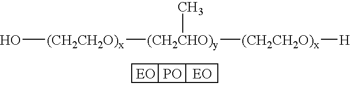

Pluronic surfactants are synthesized in a two-step process:

1. A hydrophobe of the desired molecular weight is created by the controlled addition of propylene oxide to the two hydroxyl groups of propylene glycol; and

2. Ethylene oxide is added to sandwich the hydrophobe between hydrophilic groups.

In Pluronic F68, the percentage of polyoxyethylene (hydrophile) is 80%, and the molecular weight of the hydrophobe (polyoxypropylene) is approximately 1967 Da.

Typical properties of Pluronic F68 are listed below:

Average Molecular Weight: 8400;

Melt/pour point: 52° C.;

Physical Form @ 20° C.: solid;

Viscosity (Brookfield) cps: 1000 [liquids at 25° C., pastes at 60° C. and solids at 77° C.];

Surface tension, dynes/cm at 25° C.;

0.1% Conc.: 50.3

0.01% Conc.: 51.2

0.001% Conc.: 53.6

Interfacial tension, dynes/cm at 25° C. vs Nujol;

0.1% Conc.: 19.8

0.01% Conc.: 24.0

0.01% Conc.: 26.0

Draves Wetting, Seconds 25° C.

1.0% Conc.: >360

0.1% Conc.: >360

Foam Height

Ross Miles, 0.1%, mm at 50° C.: 35

Ross Miles, 0.1%, mm at 26° C.: 40

Dynamic, 0.1%, mm at 400 ml/min: >600

Cloud point in aqueous solution, ⁰C
1% Conc.: >100
10% Conc.: >100

HLB (hydrophile-lipophile balance): 29

Other polymers having properties simlar to Pluronic F68 may also be used in the formulations of the invention.

The person skilled in the art will appreciate that one or more further surfactants may be used in addition to polyethylene-polypropylene glycol.

The formulation of the invention further comprises a stabilizing agent. A stabilizing agent may also be called an isotonicity agent.

An “isotonicity agent” is a compound that is physiologically tolerated and imparts a suitable tonicity to a formulation to prevent the net flow of water across cell membranes that are in contact with the formulation. Compounds such as glycerin, are commonly used for such purposes at known concentrations. Other suitable isotonicity agents include, but are not limited to, amino acids or proteins (e.g., glycine or albumin), salts (e.g., sodium chloride), and sugars (e.g., dextrose, sucrose and lactose).

Stabilizing (stabilizer) or isotonicity agents that maybe preferably used in accordance with the present invention include non-reducing sugars, including sucrose, trehalose, sorbose, melezitose and raffinose. Mannitol, xylitol, erythritol, threitol, sorbitol and glycerol.

In a preferred embodiment, the stabilizer or isotonicity agent is sucrose.

In a further preferred embodiment, the formulation has sucrose in a concentration ranging from 10 mg/ml to 100 mg/ml or 20 mg/ml to 80 mg/ml or about 60 mg/ml.

The formulation of the invention further comprises a citrate buffer. A citrate buffer that may be used within the present invention may e.g. be sodium citrate.

The term “buffer” or “physiologically-acceptable buffer” refers to solutions of compounds that are known to be safe for pharmaceutical or veterinary use in formulations and that have the effect of maintaining or controlling the pH of the formulation in the pH range desired for the formulation. Acceptable buffers for controlling pH at a moderately acidic pH to a moderately basic pH include, but are not limited to, such compounds as phosphate, acetate, citrate, arginine, TRIS, and histidine. “TRIS” refers to 2-amino-2-hydroxymethyl-1,3,-propanediol, and to any pharmacologically acceptable salt thereof.

It is preferred that the formulation comprises citrate in a concentration ranging from 1 to 100 mM or from 5 to 50 mM or from 10 to 20 mM.

In accordance with the present invention, it is preferred that the pH of the formulation is in the range of 5 to 7 or 5.5 to 6.5 or at or about 6. More preferably, the pH is 5.9.

The formulation of the invention may further comprise an aqueous diluent.

The term “aqueous diluent” refers to a liquid solvent that contains water. Aqueous solvent systems may be consist solely of water, or may consist of water plus one or more miscible solvents, and may contain dissolved solutes such as sugars, buffers, salts or other excipients.

The formulation may also comprise one or more non-aqueous solvents. Commonly-used non-aqueous solvents are the short-chain organic alcohols, such as, methanol, ethanol, propanol, short-chain ketones, such as acetone, and poly alcohols, such as glycerol.

The formulation of the invention preferably further comprises a preservative. Addition of a preservative is especially preferred if growth hormone is intended for multi-dose administration.

A “preservative” is a compound, which can be included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol.

A preservative may also be a bacteriostatic herein. The term “bacteriostatic” refers to a compound or compositions added to a formulation to act as an anti-bacterial agent. A preserved GH containing formulation of the present invention preferably meets statutory or regulatory guidelines for preservative effectiveness to be a commercially viable multi-use product. Examples of bacteriostatics include phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal.

Preferable, the preservative is present in a concentration ranging from 1 to 10 mg/ml or 2 to 5 mg/ml or 3 mg/ml.

The preferred preservative of the invention is phenol.

In a second aspect, the invention relates to a process for production of the liquid formulation comprising the step of preparing an aqueous solution of the components of the formulation in accordance with the present invention.

The invention further relates to a process for production of the liquid formulation comprising the step of placing a predetermined amount of the formulation into a sterile container. Typically, such an amount is in the milliliter range.

Liquid formulations of hGH for therapeutic administration may also be prepared by combining hGH and stabilizing agents having the desired degree of purity with physiologically acceptable excipients, buffers or preservatives (Remington's Pharmaceutical Sciences, 16th Edition, Osoll A. Ed (1980). Acceptable excipients are those, which are nontoxic to the patient at the concentrations and dosages employed, and include e.g. buffers, preservatives, antioxidants, pH and tonicity modifiers.

The liquid formulation of growth hormone may also include one or more other stabilizing excipients if desired. Additional stabilizing excipients may include, for example, amino acids such as glycine or alanine, mannitol or other sugar alcohols, or glycerol. In addition, the liquid formulation may include other growth factors such as insulin-like growth factors or IGF-binding proteins.

The increased stability of hGH provided by the formulation prepared in accordance with the present invention permits a wider use of hGH formulations that may be more concentrated than those commonly in use in the absence of stabilizing agents. For example, hGH liquid formulations also reduce the incidence of surface induced denaturation of hGH that occurs during aerosolisation or needleless injection of an hGH formulation.

The term “stability” refers to the physical, chemical, and conformational stability of formulations of growth hormone of the present invention (including maintenance of biological potency). Instability of a protein formulation may be caused by chemical degradation or aggregation of the protein molecules to form higher order polymers, by deglycosylation, modification of glycosylation, oxidation or any other structural modification that reduces at least one biological activity of a GH polypeptide included in the present invention.

Auto-injectors are known in the art, such as the one called Easyject®, which is particularly useful for administration of hGH. Needle-free administration may also be used in connection with the present invention, using special devices that are known in the art.

A further aspect of the invention relates to a pharmaceutical composition comprising the formulation of the invention. Compositions within the scope of this invention include all composition comprising at least one human growth hormone or derivative, analog, or variant thereof according to the present invention in an amount effective to achieve its intended purpose. While individual needs vary, determination of optimal ranges of effective amounts of each component is within the skill of the art. Typical dosages comprise about 0.001 to about 0.1 mg/kg body weight per day. When administered to patients, the hGH therapy may be administered concomitantly with other therapies which may be indicated in this disease.

The term “administer” or “administering” means to introduce a formulation of the present invention into the body of a patient in need thereof to treat a disease or condition.

In a preferred embodiment of the invention, hGH is administered in a daily dosage of about 0.1 to 10 mg or about 0.5 to 6 mg. Further preferred is a dosage of about 1 mg of human growth hormone per person per day.

In a further preferred embodiment, hGH is administered at alternating dosages, the first dosage being higher than the second dosage. Preferably, the first dosage is about 1 mg and the second dosage is about 0.5 mg. Weekly dosages are preferably about 6 mg or about 5 mg or about 4.5 mg, depending on the needs of the patient.

The term “patient” means a mammal that is treated for a disease or condition. Patients are of, but not limited to, the following origin, human, ovine, porcine, equine, bovine, rabbit and the like.

The formulation of the present invention is suitable for many different administration regimens. For example, administration can be by parenteral, such as subcutaneous, intravenous, intramuscular, oral, intraperitoneal, aerosol, transdermal, intrathecal, or rectal routes. The dosage administered depends upon the age, health and weight of the recipient, type of previous or concurrent treatment, if any, frequency of the treatment and the nature of the effect desired.

In accordance with the present invention, preferred administration routes are the subcutaneous and the intramuscular routes. A further peferred route of administration is the oral route.

It is understood that the suitable dose of a composition or formulation according to the present invention will depend upon the age, health and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This typically involves adjustment of a standard dose, e.g., reduction of the dose if the patient has a low body weight.

The total dose required for each treatment may be administered in multiple doses (multi-dose) or in a single dose (“mono-dose”).

The expression “multi-dose use” is intended to include the use of a single vial, ampoule or cartridge of GH formulation for more than one injection, for example 2, 3, 4, 5, 6 or more injections. The injections may be spaced in time, for example, by a period of 6, 12, 24, 48 or 72 hours.

Therefore, the invention further relates to the use of a formulation in accordance with the present invention for mono-dose administration. In an alternative aspect, the invention relates to the use of a formulation in accordance with the present invention for multi-dose administration. Typical amounts of hGH formulated according to the invention are 8 or 10 or 12 mg/ml for mono-dose administration, or 0.8 or 2 or 4 mg/ml for multi-dose administration.

The compositions may be administered alone or in conjunction with other therapeutics directed to the disease or directed to other symptoms thereof.

hGH formulations of the present invention may be dispensed into vials. The term “vial” refers broadly to a reservoir suitable for retaining GH in solid or liquid form in a contained sterile state. Examples of a vial as used herein include ampoules, cartridges, blister packages, or other such reservoir suitable for delivery of the GH to the patient via syringe, pump (including osmotic), catheter, transdermal patch, pulmonary or transmucosal spray. Vials suitable for packaging products for parenteral, pulmonary, transmucosal, or transdermal administration are well-known and recognized in the art.

The increased stability of hGH formulations permits long term storage at an appropriate temperature, such as below freezing (e.g. at −20° C.), or above freezing, preferably at 2-8° C., most preferably at +5° C., or even at room temperature, e.g. at +25° C.

Formulations of hGH to be used for in vivo administration must be sterile. This may e.g. be readily accomplished by filtration through sterile filtration membranes.

Therapeutic hGH liquid formulations generally are placed into a container having a sterile access port, for example, an intravenous solution bag or vial having a stopper which can be pierced by a hypodermic injection needle.

Therefore, a further aspect of the invention relates to a form of presentation of the liquid formulation of the invention hermetically closed in a sterile condition within a container suited for storage before use.

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning an range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following example of an exemplary clinical study outline, that is provided by way of illustration, and not intended to be limiting of the present invention.

### EXAMPLES

### Example 1

### Characterization of Degradation of hGH

Since the Riggin test is not specific for each degradation form (a single peak can contain more than one degradation product), an extensive characterization of the degradation profile of r-hGH liquid formulation was performed.

Strategy

The strategy of the study was divided into two phases, as described below:

First Phase:

1. Isolation of all peaks from the Riggin's method, from a developmental liquid formulation stored six months at 25° C., batch PDOI/573D6H.

2. Characterization of the peaks by mass-spectrometry of the whole molecule

3. Characterization of most abundant peaks by peptide mapping coupled to mass spectrometry.

Second Phase:

1. The peaks characterized in the first phase of the study were employed to develop alternative chromatographic methods, each specific for a given degradation route (one for desamido forms, one for oxidised forms, one for N-truncated forms, etc.).

2. After set up of the methods, these were employed to assess the content of each degradation product in two r-hGH liquid formulation samples, both stored at 4° C. as well as submitted to stressing conditions by storing them 6 days at 40° C.:
Saizen Liquid Lot SJC201 6 Days at 40° C.
Saizen Liquid Lot SJC201 18 Months at 4° C.
Serostim monodose Liquid Lot E4C101 6 Days at 40° C.
Serostim monodose Liquid Lot E4C101 18 Months at 4° C.

These samples were analysed against a freeze-dried formulation close to expiry date, Saizen freeze dried Lot B821A.

The batches were also tested by peptide mapping coupled to mass spectrometry, in order to fully characterize the molecules.

3. From the data obtained the degradation pathway was established.

Results

The characterisation allowed to assess that the degradation forms of r-hGH are as follows:
Iso-Asp 130 (major) and Iso-Asp107 (secondary), deriving from isomerisation of Aspartic acids.
Deamidation at either Asn 149 or 152, and at Asn 99
Succinimide intermediates of either Asp isomerisation or Asn deamidation (mainly succinimide of Asp 130).
Des-Phe-Pro r-hGH deriving from the loss of the two N-terminal amino acids
Oxidation, mainly at methionine 14.
The following methods were developed:
RP-HPLC in TFA for oxidised forms
Ion-exchange chromatography for desamido forms
Peptide mapping for both iso-Asp 130 and iso-Asp 107
HI-HPLC for Des-Phe-Pro r-hGH
Whereas the succinimide forms are quantifiable by Riggin's (major one is succinimide of Asp 130).

The amount of degradation forms detected in the samples are shown in Table A. By comparing the data above, it can be stated that the major degradation routes of r-hGH in the liquid formulations are:
Deamidation
Isomerisation of Aspartic acids
Loss of the two N-terminal amino acids.

Oxidation does not increase upon storage at 40° C. for 6 days.

TABLE A
Quantification of degraded Forms in r-hGH formulations

| | Amount in | Amount in | Amount in Saizen | Amount in Serostim | Amount in Saizen |
|---|---|---|---|---|---|
| Degradation | Saizen | Serostim | SJC101 stored 6 | E4C101 stored 6 | B821A |
| Form | SJC101 | E4C101 | days at 40° C. | days at 40° C. | (freeze-dried) |
| Desamido | 1.0% | 1.5% | 11.0% | 8.0% | 0.3% |
| Iso-Asp 130 | 3.5% | 3.5% | 12.5% | 13.9% | 1.8% |
| Iso-Asp107 | 2.4% | 3.4% | 5.0% | 5.0% | 3.1% |
| Succinimide | 3.0% | 2.0% | 3.0% | 1.3% | 0.1% |
| 130 | | | | | |
| Met- | 0.2% | 0.2% | 0.2% | 0.2% | 0.1% |
| sulphoxide | | | | | |
| Des-Phe-Pro | 2.6% | 2.7% | 4.3% | 5.0% | 2.3% |
| hGH | | | | | |
| Split 130/131 | ND | ND | ND | ND | ND |
| Purity by | 87% | 87% | 64% | 67% | 92% |
| sum of | | | | | |
| degradation | | | | | |
| Purity by | 90% | 90% | 78% | 74% | 96% |
| Riggin's | | | | | |

### Example 2

### hGH Liquid Formulations for Mono-Dose Administration

Introduction

A liquid formulation of r-hGH was developed, which presents a simpler pharmaceutical presentation than a lyophilized product. A tiered development approach was taken to ensure the safety of the new liquid formulation of r-hGH. Extensive characterization of the degradation profile was performed as well as studies of general toxicity and local tolerability conducted for this new formulation. In addition a comparative bioavailability study was performed to demonstrate bioequivalence between the new liquid formulation and the currently approved lyophilized formulation.

As part of the pharmaceutical development, the suitability and stability of the formulation was assessed for the following parameters:
pH and buffers
Ionic strength of the buffer
Excipients

The stability of the candidate formulations was then studied in the container of choice to determine the manufacturing process, overfill and container closure system.

Following this development the drug product was presented as an aqueous solution of recombinant human growth hormone in citrate buffer at pH 5.85, Sucrose and Poloxamer 188 filled into 3 ml glass cartridges. The drug product was formulated at a concentration of 12.0 mg/ml and supplied in a 3 ml nominal capacity cartridge with bromobutyl stopper. The cartridges are filled with 0.5 ml of r-hGH solution for injection in order to deliver 6.0 mg of r-hGH to the patient.

Components of the Drug Product

The drug product was presented as a mono-dose aqueous solution of Somatropin, (recombinant human Growth Hormone, r-hGH) in Citrate Buffer, Sucrose and Poloxamer 188 filled into 3 ml type I glass cartridge.

The drug product was formulated at a concentration of 12.0 mg/ml in order to deliver 6.0 mg/dose of r-hGH to the patients when 0.5 ml of solution is administered.

Drug Substance

Compatibility of the Drug Substance with Formulation Excipients

The compatibility of the drug substance with the formulation excipients was evaluated taking into consideration experiments done during the development of the r-hGH lyophilized formulation as well as during preliminary screening studies aimed to investigate the feasibility of a liquid product presentation. These experiments showed that:
The r-hGH main degradation product is the deamidated form and this is strictly correlated to the pH. A pH around 6.0 minimizes such degradation;
The solubility is an additional critical parameter influenced by pH, presence of bacteriostatic agents and mechanical stress. In order to avoid precipitation of r-hGH, it is necessary to find an appropriate equilibrium among the formulation components.

Selection of Buffering Agent

The selection of the buffering agent was evaluated taking into account the following:
Effect of the buffering agent concentration on the deamidation rate;
Effect of the buffering agent concentration on the protein solubility;
Effect of the buffering agent concentration on the pH stability.

Several solutions containing the drug substance and the buffer (acetate and citric acid) were manufactured and submitted to shaking stressing tests in order to induce r-hGH precipitation; the same solutions were also stored at +25±2° C. in order to accelerate the degradation pathway.

Results showed the following:
The buffering agent concentration has a relevant impact on r-hGH deamidation; in particular the higher the concentration, the higher is the degradation;
The buffering agent concentration has a relevant impact on r-hGH solubilisation; in particular the higher the concentration, the lower is the solubility;
The use of citric acid as acidifying agent in comparison to a 10 mM acetate buffer better stabilises the pH close to 6.0.

Selection of Excipients

The compatibility of the drug substance with two different excipients (Sucrose and Mannitol) was evaluated assessing their impact on the r-hGH solubility and deamidation rate.

Several solutions containing buffering system and Sucrose or Mannitol, were manufactured and submitted to shaking stressing tests in order to induce r-hGH precipitation; the same solutions were also stored at +25±2° C. in order to accelerate the degradation pathway.

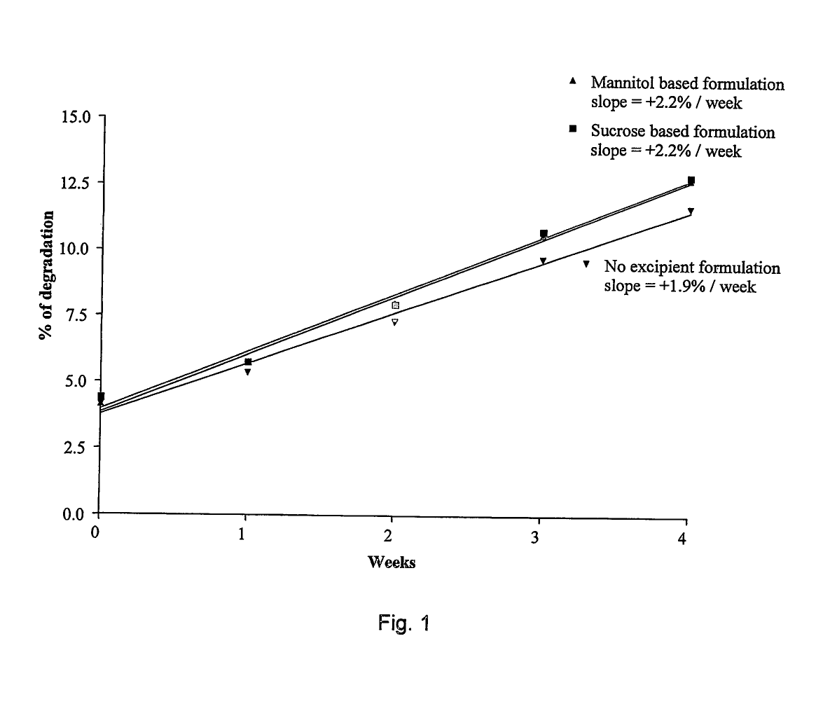

Experimental results showed that:
Either Sucrose or Mannitol improves the r-hGH solubility;
The addition of Mannitol or Sucrose to the formulation slightly increases the degradation of r-hGH tested by RP-HPLC (FIG. 1).

FIG. 1 shows the RP-HPLC results after 4 weeks storage at +25±2° C. of r-hGH liquid formulations containing Mannitol or Sucrose or no excipient.

Selection of a Surfactant

Several formulations containing buffering system, isotonicity agent (Sucrose or Mannitol) and the surfactants (tensioactive agent) under evaluation (Polysorbate 20 or Poloxamer 188) were manufactured and submitted to shaking stressing tests (mechanical stress test performed by using a Vortex) which induces r-hGH precipitation. Two concentrations of surfactant were tested during this preliminary assessment: one high concentration of 1.0 mg/ml and one low concentration of 0.01 mg/ml. Subsequently the formulations were visually inspected. The same solutions were stored at +25±2° C. in order to accelerate the degradation pathway.

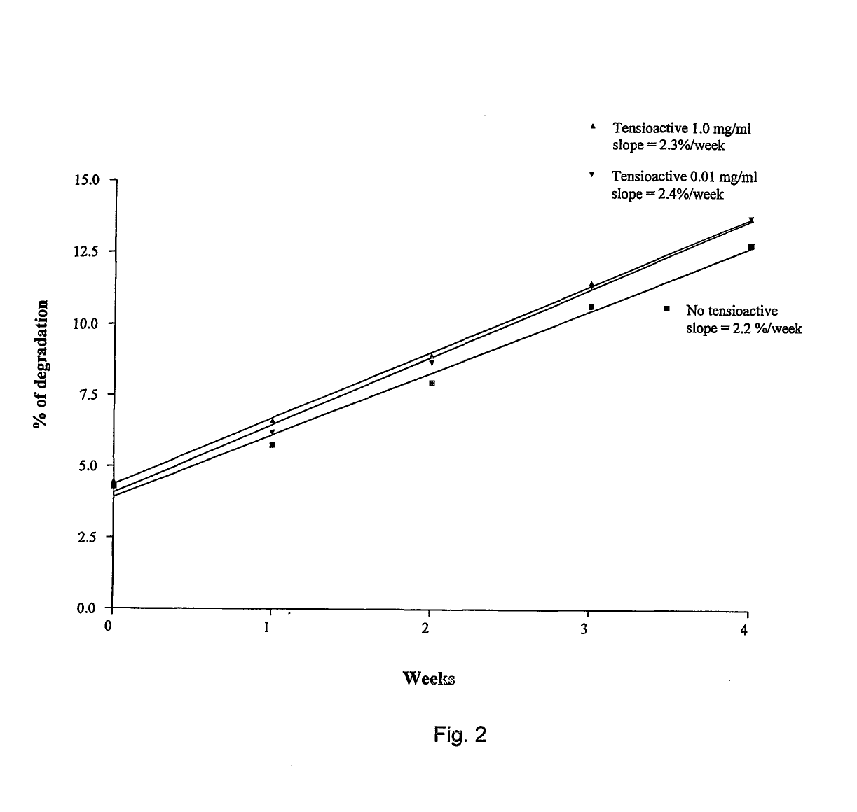

Results showed the following:
The higher tensioactive agent concentration tested (1.0 mg/ml) significantly improves the protein solubility even if, some positive effect was observed adding a lower amount such as 0.01 mg/ml;
The addition of a tensioactive agent (Poloxamer 188 or Polysorbate 20 at 1.0 or 0.01 mg/ml) to the formulation slightly increases the degradation of r-hGH (FIG. 2);
Poloxamer 188 and Polysorbate 20 equally improve r-hGH solubility.

FIG. 2 shows the RP-HPLC results after 4 weeks storage at +25±2° C. of r-hGH liquid formulations containing tensioactve at various concentrations and no tensioactive.

During a parallel development of a liquid multi-dose formulation, it was observed that Polysorbate 20 induces the formation of a white opalescent suspension in presence of bacteriostatic agents such as m-Cresol and Phenol. Due to this effect Poloxamer 188 was selected as preferred surfactant.

Overall Conclusion from Screening Studies:

The conclusion of the pre-formulation studies is summarized as follows:
The buffering agent concentration has an impact on r-hGH deamidation and solubilisation; (a buffer molarity greater than 20 mM induces a higher degradation of the molecule)
The use of citric acid as acidifying agent in contrast to a 10 mM acetate buffer better stabilises the pH close to 6.0;
Either Sucrose or Mannitol improve the r-hGH solubility and slightly increase the degradation of the r-hGH;
The presence of a surfactant at concentration of at least 1.0 mg/ml significantly improves the protein solubility
Poloxamer 188 and Polysorbate 20 equally improve the r-hGH solubility;
From a parallel study aimed to develop a liquid multidose presentation, incompatibility between Polysorbate 20 and bacteriostatic agents such as Phenol or m-Cresol was observed limiting its use.

Candidate Formulations

From the previous screening studies, five candidate formulations were manufactured at lab scale and submitted to accelerated (+25±2° C.) and long-term (5±3° C.) stability. One sample of each formulation was filled into 3 ml glass cartridges equipped with a halobutyl closure system.

The composition of the 5 formulations is described in the following Table 1:

TABLE 1
Composition of 5 candidate formulations

| | Candi- | Candi- | Candi- | Candi- | |
|---|---|---|---|---|---|
| | date | date | date | date | Candidate |
| Ingredient | #1 | #2 | #3 | #4 | #5 |
| r-hGH (mg/ml) | 8.0 | 12.0 | 8.0 | 12.0 | 8.0 |
| Sucrose (mg/ml) | 60.0 | 60.0 | 60.0 | 60.0 | 60.0 |
| Poloxamer 188 | 1.5 | 1.5 | 1.5 | 1.5 | 1.5 |
| (mg/ml) | | | | | |
| Citric acid | — | — | q.s. to | q.s. to | — |
| | | | pH 5.9 | pH 5.9 | |
| Acetate buffer | 10 mM | 10 mM | — | — | — |
| pH 5.9 | | | | | |
| Citric buffer | — | — | — | — | 10 mM |
| pH 5.9 | | | | | |
| WFI | q.s. to | q.s. to | q.s. to | q.s. to | q.s. to |
| | 1 ml | 1 ml | 1 ml | 1 ml | 1 ml |

Lab Scale Batches and Stability Results

The following tables 2 and 3 report the results obtained testing the formulations for the most relevant stability indicating parameters (r-hGH content, related proteins, dimers and high molecular weight substances, particulate matter and pH).

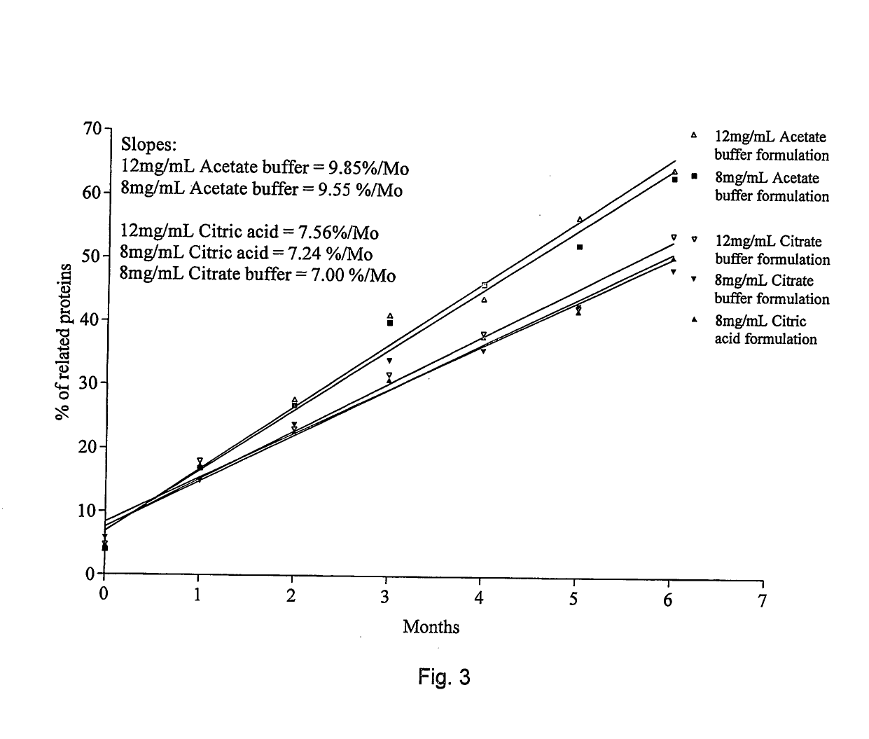

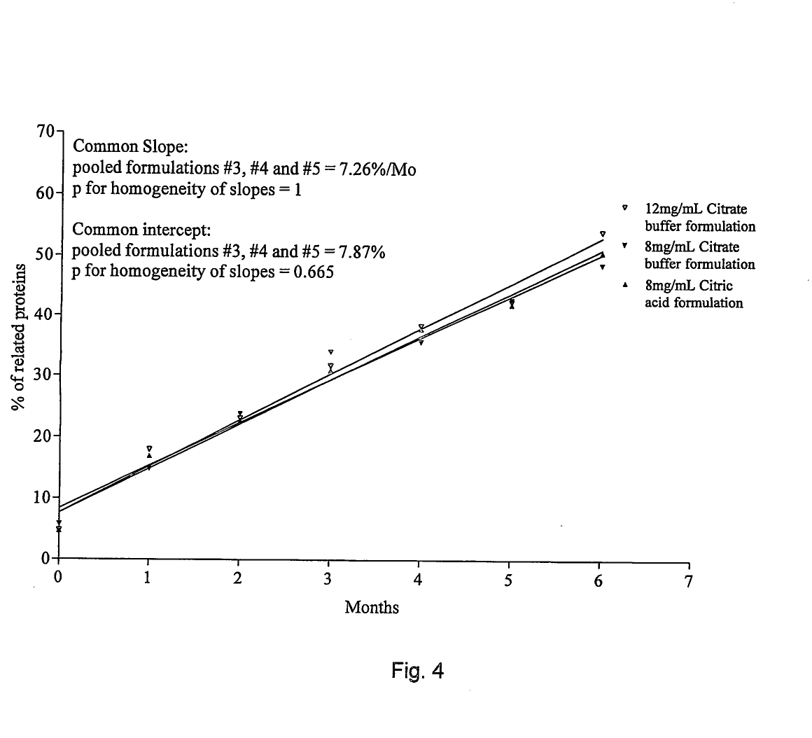

The RP-HPLC data for related proteins were statistically analyzed by linear regression. The results obtained showed the following:
The acetate buffered formulations at 8.0 and 12.0 mg/ml have a degradation rate greater than the formulations containing citric acid or citrate buffer; this excluded the use of the acetate as buffer of the r-hGH formulation (FIG. 3);
The regression lines of candidate formulation #3, #4 and #5 showed an homogenous slope and homogeneous intercept;
Citrate buffer and citric acid are similar in terms of degradation rate;
The r-hGH concentration does not play a relevant role for such degradation (FIG. 4).

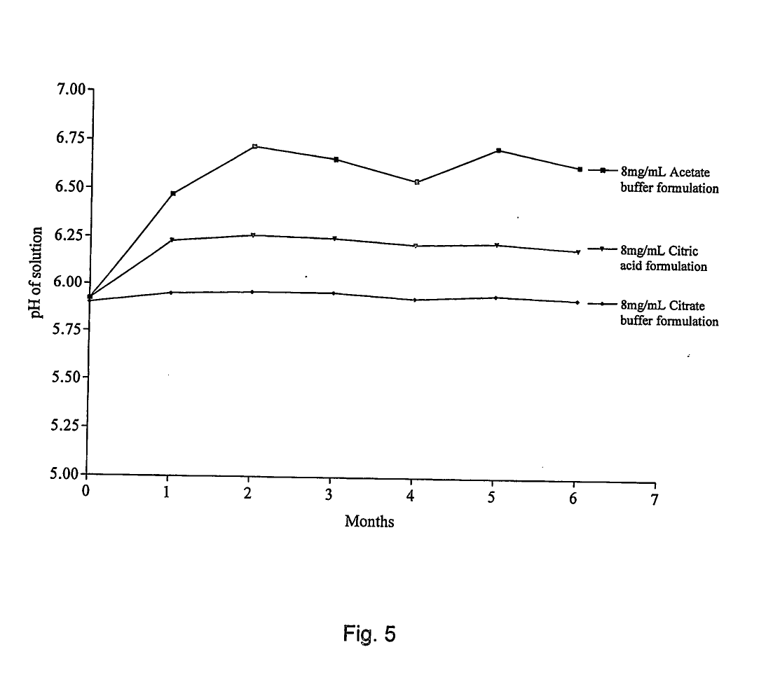

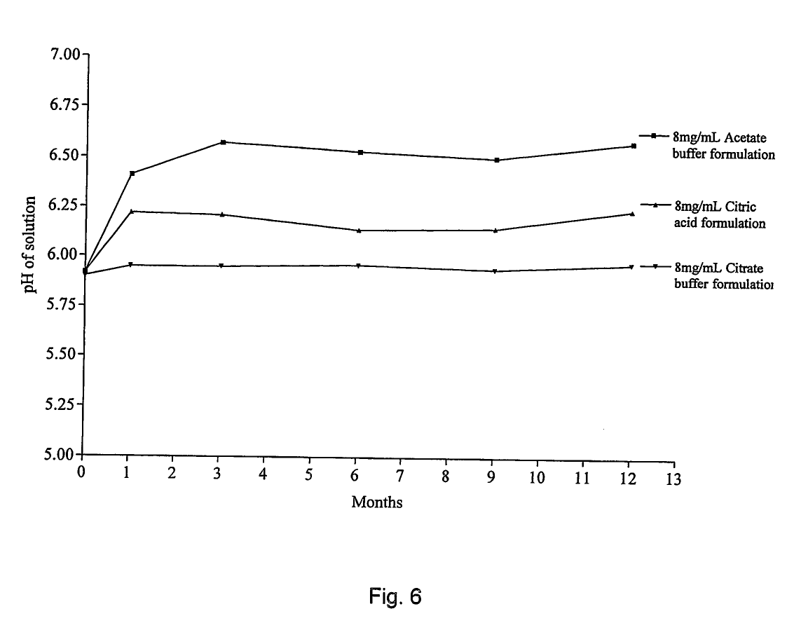

The reasons for the different degradation rate of the lab scale candidate formulations is due to the difference in pH among the formulations. The acetate buffered formulations, as well as the formulation containing citric acid as acidifying agent, show a significant increase of that parameter after 1 month storage (FIG. 5 (stability at +25° C.) and FIG. 6 (stability at +4° C.)). The relationship between the pH and the deamidation rate is well known and it is the most important parameter to control in order to reduce this degradation. Formulation with citrate buffer has a stable pH that could explain the reason of its better stability in comparison with the others.

TABLE 2
Stability data at 5 ± 3° C. of lab scale formulations

| Formulation | T = 0 | 1 Mo | 3 Mo | 6 Mo | 9 Mo | 12 |
|---|---|---|---|---|---|---|
| Candidate #1 | | | | | | |
| r-hGH content (mg/ml) | 8.61 | 7.56 | 8.01 | 8.07 | 7.85 | 7.87 |
| Related proteins (%) | 4.07 | 5.84 | 8.50 | 11.42 | 12.97 | 16.38 |
| Dimers and High molecular weight substances | 0.05 | 0.06 | 0.07 | 0.07 | 0.11 | 0.10 |
| (%) | | | | | | |
| Particulate matter | Free | — | Free | Free | Free | Free |
| PH | 5.92 | 6.41 | 6.57 | 6.53 | 6.50 | 6.58 |
| Candidate #2 | | | | | | |
| r-hGH content (mg/ml) | 12.82 | 11.70 | 12.06 | 12.10 | 12.04 | 11.79 |
| Related proteins (%) | 4.07 | 5.96 | 8.86 | 12.89 | 14.40 | 18.33 |
| Dimers and High molecular weight substances | 0.05 | 0.06 | 0.09 | 0.08 | 0.09 | 0.14 |
| (%) | | | | | | |
| Particulate matter | Free | — | Free | Free | Free | Free |
| PH | 5.92 | 6.50 | 6.52 | 6.50 | 6.54 | 6.20 |
| Candidate #3 | | | | | | |
| r-hGH content (mg/ml) | 8.65 | 8.61 | 8.27 | 8.70 | 9.13 | 8.37 |
| Related proteins (%) | 4.72 | 5.61 | 7.14 | 9.36 | 11.51 | 12.48 |
| Dimers and High molecular weight substances | 0.02 | 0.02 | 0.02 | 0.09 | 0.08 | 0.07 |
| (%) | | | | | | |
| Particulate matter | Free | — | Free | Free | Free | Free |
| PH | 5.92 | 6.22 | 6.21 | 6.14 | 6.15 | 6.24 |
| Particulate matter | Free | — | Free | Free | Free | Free |
| PH | 5.90 | 5.95 | 5.95 | 5.96 | 5.94 | 5.97 |
| Candidate #4 | | | | | | |
| r-hGH content (mg/ml) | 11.56 | 12.04 | 12.78 | 12.60 | 13.42 | 12.70 |
| Related proteins (%) | 4.78 | 6.48 | 6.38 | 10.85 | 11.64 | 13.49 |
| Dimers and High molecular weight substances | 0.02 | 0.04 | 0.03 | 0.13 | 0.08 | 0.08 |
| (%) | | | | | | |
| Particulate matter | Free | — | Free | Free | Free | Free |
| PH | 5.92 | 6.22 | 6.23 | 6.21 | 6.20 | 6.28 |
| Candidate #5 | | | | | | |
| r-hGH content (mg/ml) | 8.00 | 8.15 | 8.34 | 8.12 | 8.11 | 8.19 |
| Related proteins (%) | 5.82 | 6.49 | 9.23 | 10.29 | 11.25 | 13.22 |
| Dimers and High molecular weight substances | 0.02 | 0.01 | 0.04 | 0.07 | 0.16 | 0.06 |
| (%) | | | | | | |
| Particulate matter | Free | — | Free | Free | Free | Free |
| PH | 5.90 | 5.95 | 5.95 | 5.96 | 5.94 | 5.97 |

TABLE 3
Stability data at 25 ± 2° C. of lab scale formulations

| Formulation | T = 0 | 1 o | 2 Mo | 3 Mo | 4 Mo | 5 Mo | 6 Mo |
|---|---|---|---|---|---|---|---|
| Candidate #1 | | | | | | | |
| r-hGH content (mg/ml) | 8.61 | 7.92 | 7.98 | 8.12 | 8.23 | 8.01 | 8.20 |
| Related protens (%) | 4.07 | 16.82 | 26.85 | 39.98 | 46.09 | 52.33 | 63.09 |
| Dimers and High molecular weight | 0.05 | 0.10 | 0.14 | 0.20 | 0.25 | 0.34 | 0.37 |
| substances (%) | | | | | | | |
| Particulate matter | Free | nt | nt | Free | nt | nt | Free |
| PH | 5.92 | 6.47 | 6.72 | 6.66 | 6.55 | 6.72 | 6.63 |
| Candidate #2 | | | | | | | |
| r-hGH content (mg/ml) | 12.82 | 12.01 | 12.80 | 11.98 | 12.45 | 12.25 | 12.42 |
| Related proteins (%) | 4.07 | 17.21 | 27.76 | 41.10 | 43.89 | 56.65 | 64.36 |
| Dimers and High molecular weight | 0.05 | 0.11 | 0.16 | 0.26 | 0.29 | 0.43 | 0.51 |
| substances (%) | | | | | | | |
| Particulate matter | Free | nt | nt | Free | nt | nt | Free |
| PH | 5.92 | 6.53 | 6.79 | 6.65 | 6.50 | 6.78 | 6.66 |
| Candidate #3 | | | | | | | |
| r-hGH content (mg/ml) | 8.65 | 8.50 | 8.20 | 8.70 | 8.17 | 8.34 | 8.62 |
| Related proteins (%) | 4.72 | 16.90 | 22.66 | 30.88 | 37.76 | 41.90 | 50.57 |
| Dimers and High molecular weight | 0.02 | 0.06 | 0.13 | 0.21 | 0.36 | 0.38 | 0.57 |
| substances (%) | | | | | | | |
| Particulate matter | Free | nt | nt | Free | nt | nt | Free |
| PH | 5.92 | 6.21 | 6.25 | 6.22 | 6.24 | 6.28 | 6.25 |
| Candidate #4 | | | | | | | |
| r-hGH content (mg/ml) | 11.56 | 12.60 | 12.17 | 12.48 | 12.43 | 12.87 | 12.69 |
| Related proteins (%) | 4.78 | 17.95 | 23.06 | 31.72 | 38.28 | 42.38 | 53.98 |
| Dimers and High molecular weight | 0.02 | 0.09 | 0.18 | 0.35 | 0.47 | 0.62 | 1.06 |
| substances (%) | | | | | | | |
| Particulate matter | Free | nt | nt | Free | nt | nt | Free |
| PH | 5.92 | 6.23 | 6.26 | 6.25 | 6.22 | 6.23 | 6.20 |
| Candidate #5 | | | | | | | |
| r-hGH content (mg/ml) | 8.00 | 8.29 | 8.20 | 8.37 | 8.31 | 8.47 | 8.18 |
| Related proteins (%) | 5.82 | 14.84 | 23.81 | 34.01 | 35.64 | 42.76 | 48.57 |
| Dimers and High molecular weight | 0.02 | 0.09 | 0.16 | 0.27 | 0.34 | 0.38 | 0.53 |
| substances (%) | | | | | | | |
| Particulate matter | Free | nt | nt | Free | Nt | nt | Free |
| PH | 5.90 | 5.95 | 5.96 | 5.96 | 5.93 | 5.95 | 5.93 |

nt = not tested

In addition, the SE-HPLC for r-hGH content results were statistically analyzed by linear regression model. Results showed the following:

No statistically significant differences among the lab scale batches and no statistically significant trend was observed over the stability period. In Tables 4 and 5 the statistical results are reported.

TABLE 4
Statistical results of SE-HPLC data of lab scale formulations after 6 months storage at 25 ± 2° C.

| Candidate | Individual | Individual | p for common | Common slope | p for significativity |
|---|---|---|---|---|---|
| formulation | slope (mg/Mo) | intercept (mg) | slope (>0.05) | (mg/Mo) | of common slope (>0.05) |
| #1 | −0.029 | 8.24 | 0.08423 | +0.020 | 0.9078 |
| #2 | −0.038 | 12.50 | | | |
| #3 | −0.016 | 8.50 | | | |
| #4 | +0.150 | 11.95 | | | |
| #5 | +0.036 | 8.15 | | | |

TABLE 5
Statistical results of SE-HPLC data of lab scale formulations after 12 months storage at 5 ± 3° C.

| Candidate | Individual | Individual | p for common | Common slope | p for significativity of |
|---|---|---|---|---|---|
| formulation | slope (mg/Mo) | Intercept (mg) | slope (>0.05) | (mg/Mo) | common slope (>0.05) |
| #1 | −0.025 | 8.12 | 0.07365 | +0.01 | 0.9122 |
| #2 | −0.039 | 12.29 | | | |
| #3 | +0.01 | 8.58 | | | |
| #4 | +0.101 | 12.00 | | | |
| #5 | +0.004 | 8.13 | | | |

Moreover, no differences between candidate formulations were observed for the parameter particulate matter.

Conclusion

The stability study on the 5 candidate formulations confirmed the important role of the pH for the control of the r-hGH degradation rate. The following formulation was therefore selected for the production at pilot scale:

| Component | Amount | |
|---|---|---|
| r-hGH | 12.0 | mg |
| Poloxamer P188 | 1.5 | mg |
| Sucrose | 60.0 | mg |
| Citrate buffer pH 5.85 | 10 | mM |

| | |
|---|---|
| WFI | q.s. to 1 ml |

Excipients

The formulation development described above has determined the excipients required to stabilise a liquid r-hGH formulation. These excipients are described below to explain:
the function
the selected concentration
the compliance with Pharmacopoeial monographs

Sucrose

The function of Sucrose is to create an isotonic environment and stabilise the protein. The concentration of Sucrose selected for the finished product is 60.0 mg/ml.

The Sucrose used complies with USP and Ph. Eur.

Citrate Buffer

10 mM citric acid is adjusted to pH 5.8-6.2 by addition of Sodium Hydroxide solution reducing the formation of deamidated protein whilst maintaining adequate solubility.

Citrate buffer complies with Ph. Eur., and USP.

Poloxamer 188

Poloxamer 188 is used to increase the solubility of r-hGH and to avoid the formation of particulate matter.

The concentration of 1.5 mg/ml was selected since it is the most effective in enhancing the solubility of such protein.

Poloxamer 188 used complies with the USP and Ph. Eur.

Water for Injections

All the excipients are fully soluble in WFI at the required concentrations.

The WFI used complies to the USP and Ph. Eur.

### Example 4

### Formulation Development

After appropriate preformulation studies and assessment of the stability results available on lab scale batches, the final formulation was selected.

Physico-Chemical and Biological Properties

Effect of pH

Based on the studies performed to determine the effects of the pH, it was observed that the pH is a significant parameter for the r-hGH stability. In particular, a pH range between 5.8 to 6.2 is able to minimize the deamidation, which is one of the main degradation routes of r-hGH.

Effect if Ionic Strength

The experiments performed demonstrated that the increased molarity of the buffer system present in the liquid formulation might induce a higher degradation rate detected by RP-HPLC and as well as reduce the r-hGH solubility and induce precipitation. The buffering system was therefore selected at the concentration of 10 mM to optimise the pH whilst minimizing negative effects on the r-hGH stability.

Aggregation (Dimers and HMW substances)

The experimental studies performed showed that r-hGH presented in liquid formulation is unlikely to form aggregates during the storage at accelerated (+25±2° C.) or long-term (+5±3° C.) conditions.

Biological Activity

In-vivo biological activity (weight gain in hypophysectiomized rats) was tested by Bioassay. The specific activity results for both the newly developed liquid formulation as well as the lyophilised formulation met the established acceptance criteria for specific activity.

Degradation Profile

Extensive characterisation was performed to compare the degradation profile of the newly developed liquid formulation with the degradation profile of the currently approved lyophilised formulation. Results obtained demonstrated that the same degradation profile of r-hGH was obtained during storage, with higher amounts of related proteins identified by RP-HPLC for the liquid formulation.

### Example 5

### Manufacturing Process Development

The manufacturing process of this liquid formulation consists of the following steps:
Compounding of the liquid formulation;
Pre-filtration through 0.45 μm membrane
Sterile filtration through 0.22 μm membrane
Filling into the cartridge
Crimping of each cartridge

This is a standard manufacturing process (see also flow chart on the following page) for this pharmaceutical form. The most critical process step is the sterilization by aseptic filtration. During the development of the liquid mono-dose formulation, it was observed that, the compounded drug product solution presented some slight opalescence before filtration. This opalescence is probably due to some precipitation of native r-hGH related to the low solubility of this molecule at a slight acidic pH. In order to avoid that the sterile filtration through a 0.22 μm membrane represents a difficult and risky step during the drug product manufacturing process, a pre-filtration through a 0.45 μm membrane was added.

[image]

A study was therefore performed with the purpose to investigate whether the addition of a pre-filtration step was suitable to clarify the solution before sterile filtration taking into account constraints related to the manufacturing equipment that were:
Filtration ratio not more than 15 g/cm2
Nitrogen pressure not greater than 1.5 atm.

The composition of the formulation used to investigate the pre-filtration step is reported in Table 6.

TABLE 6
Composition of formulation for investigation
of the pre-filtration step

| Component | Amount | |
|---|---|---|
| r-hGH | 12.0 | mg |
| Poloxamer P188 | 1.5 | mg |
| Sucrose | 60.0 | mg |

| | |
|---|---|
| Citric acid | q.s. to pH 5.9 |
| WFI | q.s. to 1 ml |

The study results are as follows:
17 g/cm2 of compounded can be filtered on 0.45 μm membrane at 1 atm.
31 g/cm2 of pre-filtered can be filtered on 0.221 μm membrane at 1 atm.

The study concluded that, the introduction of a pre-filtration step using a 0.45 μm membrane, with a Nitrogen pressure of 1 atm, is suitable to clarify the r-hGH liquid compounded solution solution before sterile filtration through a 0.22 μm membrane. The filtration ratio of 15 g/cm² proposed by the drug product manufacturer is also suitable to perform the pre-filtration or the sterile filtration.

The following flow chart summarizes the pilot-scale manufacturing process:

### Example 6

### Development of Liquid GH Formulation for Multi-Dose Administration

Compatibility of the Drug Substance with Excipients

The results are here below summarized:
The r-hGH main degradation product is the de-amidated form and this is correlated to the pH. pH values around 6.0 minimize such degradation;

The solubility is an additional critical parameter influenced by pH, presence of bacteriostatic agents and mechanical stress.

In order to avoid precipitation of r-hGH, it is necessary to find an appropriate equilibrium among the formulation components.

Selection of Buffering Agent

The selection of the buffering agent to add in the liquid formulation was evaluated taking into account the following:

Effect of the buffering agent concentration on the deamidation rate;

Effect of the buffering agent concentration on the protein solubility;

Effect of the buffering agent concentration on the pH stability.

Several solutions containing the drug substance at 6.0 and 12.0 mg/mL and the buffer (acetate and citric acid) were manufactured and submitted to shaking stressing tests in order to induce r-hGH precipitation; the same solutions were also stored at +40±2° C. in order to accelerate the degradation pathway.

Results showed the following:
The buffering agent concentration has a relevant impact on r-hGH deamidation; in particular the higher is the molarity, the higher is the degradation;
The buffering agent concentration has a relevant impact on r-hGH solubilisation; in particular the higher is the molarity, the lower is the solubility;
The use of citric acid as acidifying agent respect to a 10 mM acetate buffer better stabilises the pH close to 6.0.
   Selection of Isotonicity agents

The compatibility of the drug substance with two different isotonicity agents (Sucrose and Mannitol) was evaluated assessing their impact on the r-hGH solubility and deamidation rate.

Several solutions containing buffering system at 6.0 and 12.0 mg/mL and Sucrose or Mannitol, were manufactured and submitted to shaking stressing tests in order to induce r-hGH precipitation; the same solutions were also stored at +40±2° C. in order to accelerate the degradation pathway.

Results showed the following:
Either Sucrose or Mannitol improve the r-hGH solubility;
The addition of Mannitol or Sucrose to the formulation slightly increases the degradation of the r-hGH.
   Selection of a Surfactant

Several formulations at 6.0 and 12.0 mg/mL were manufactured and submitted to shaking stressing tests in order to induce r-hGH precipitation; the same solutions were also stored at +25±2° C. in order to accelerate the degradation pathway.

Results showed the following:
The higher surfactant concentration tested (1.0 mg/ml) significantly improve the protein solubility even if, some positive effect was observed adding a lower amount such as 0.01 mg/ml;
The addition of surfactant to the formulation slightly increase the degradation of the r-hGH;
Poloxamer 188 and Polysorbate 20 equally improve the r-hGH solubility.
   Selection of a Bacteriostatic Agent

Several formulations at 0.8 mg/mL of r-hGH containing 0.9% Benzyl alcohol or 0.3% Phenol were manufactured and submitted to bacteriostatic efficacy screening study. M-Cresol was not tested due to its incompatibility with surfactants.

Results showed the following:
Both 0.9% Benzyl alcohol and 0.3% Phenol met the criteria B of Eu. Ph.
   Overall Conclusion from Pre-Formulation Studies

The conclusion of the pre-formulation studies are summarized as follows:
The buffering agent concentration has an impact on r-hGH deamidation and solubilisation;
The use of citric acid as acidifying agent respect to a 10 mM acetate buffer better stabilises the pH close to 6.0;
Either Sucrose or Mannitol improve the r-hGH solubility and slightly increase the degradation of the r-hGH;
The presence of a surfactant at concentration of at least 1.0 mg/ml significantly improve the protein solubility even if, some positive effect was observed adding a lower amount such as 0.01 mg/ml;
The addition of tensioactive to the formulation slightly increase the degradation of the r-hGH;
Poloxamer 188 and Polysorbate 20 equally improve the r-hGH solubility;
M-Cresol is not compatible with surfactants;
0.9% Benzyl alcohol and 0.3% Phenol met criteria B of Eu. Ph.
   Candidate Formulations Stability Studies

From the previous pre-formulation studies, 8 candidate formulations at 0.8 mg/mL and 4.0 mg/mL of r-hGH containing both acetate and citric acid as buffer agent and Benzyl alcohol or Phenol as bacteriostatic agent were manufactured at lab scale and submitted to accelerated (+25±2° C.) and long-term (5±3° C.) stability. Samples of each formulation were filled into 3 mL glass cartridges equipped with a West Pharmaceutical bromobutyl 4023/50 plungers. During the stability of this first set of 8 candidates, it was decided to manufacture one additional formulation at 0.8 mg/mL containing 10 mM citrate buffer and Phenol. The composition of the 9 formulations is described in the following Tables.

TABLE 7
Composition of candidate formulations

| | HGH/BA/ | HGH/BA/ | HGH/BA/ | HGH/BA/ |
|---|---|---|---|---|
| Ingredient | acetic/0.8 mg | acetic/4 mg | citric/0.8 mg | citric/4 mg |
| r-hGH | 0.8 | 4.0 | 0.8 | 4.0 |
| (mg/g) | | | | |
| Sucrose | 60.0 | 60.0 | 60.0 | 60.0 |
| (mg/g) | | | | |
| Poloxamer | 1.5 | 1.5 | 1.5 | 1.5 |
| 188 | | | | |
| (mg/g) | | | | |
| Citric acid | — | — | q.s. to | q.s. to |
| | | | pH 5.9 | pH 5.9 |
| Acetate | 10 mM | 10 mM | — | — |
| buffer | | | | |
| pH 5.9 | | | | |
| Citric | — | — | — | — |
| buffer | | | | |
| pH 5.9 | | | | |
| Benzyl | 9.0 | 9.0 | 9.0 | 9.0 |
| alcohol | | | | |
| (mg/g) | | | | |

TABLE 8
Composition of candidate formulations

| | HGH/PHE/ | HGH/PHE/ | HGH/PHE/ | HGH/PHE/ | HGH/PHE/CitrBuff |
|---|---|---|---|---|---|
| Ingredient | acetic/0.8 mg | acetic/4 mg | citric/0.8 mg | citric/4 mg | 0.8 mg/WEST |
| r-hGH | 0.8 | 4.0 | 0.8 | 4.0 | 0.8 |
| (mg/g) | | | | | |
| Sucrose | 60.0 | 60.0 | 60.0 | 60.0 | 60.0 |
| (mg/g) | | | | | |
| Poloxamer | 1.5 | 1.5 | 1.5 | 1.5 | 1.5 |
| 188 | | | | | |
| (mg/g) | | | | | |
| Citric acid | — | — | q.s. to pH | q.s. to pH | — |
| | | | 5.9 | 5.9 | |
| Acetate | 10 mM | 10 mM | — | — | — |
| buffer | | | | | |
| pH 5.9 | | | | | |
| Citric | — | — | — | — | 10 mM |
| buffer | | | | | |
| pH 5.9 | | | | | |
| Phenol | 3.0 | 3.0 | 3.0 | 3.0 | 3.0 |
| (mg/g) | | | | | |

Candidate Formulations Stability Results

Stability data of candidate formulations are available up to six months accelerated conditions at +25±2° C. and 12 months long-term at +5±3° C. (see enclosed tables).

Results show the following:
Formulations containing acetate buffer are less stable than formulations containing citric acid or citrate buffer;
Formulation containing citrate buffer better stabilises the pH of the solution;
The only degradation pathway observed over the stability period is the deamidation of r-hGH detectable by RP-HPLC;
Formulations containing 0.9% Benzyl alcohol showed non-homogeneous behaviour and for this reason were discharged. This is probably related to impurities present in different batches of Benzyl alcohol;
The protein concentration does not impact the degradation rate;
The estimated degradation rate at +5±3° C. of the citrate buffered and citric acid formulation are comparable (0.7-0.8%/month), while formulation containing acetate buffer has a degradation rate of about 1.1%/month.

On the basis of the above results it was decided to select the formulation containing Citrate buffer and Phenol for the technology transfer to the manufacturing site.

### Example 7

### Specific Formulation for Multi-Dose Administration

The following formulations for multi-dose administration were tested for stability and solubility

Formulations 6 to 8 (for multi-dose administration) were as depicted in Table 9:

TABLE 9

| | 6 | 7 | 8 |
|---|---|---|---|
| r-hGH (mg/ml) | 9.3 | 9.3 | 9.3 |
| Sucrose (mg/ml) | 60.0 | 60.0 | 60.0 |
| Benzyl alcohol (mg/ml) | 9.0 | — | — |
| Phenol | | 5.0 | 2.5 |
| Pluronic F68 (mg/ml) | 2.0 | 2.0 | 2.0 |
| Citric acid | to pH 6.0 | to pH 6.0 | to pH 6.0 |

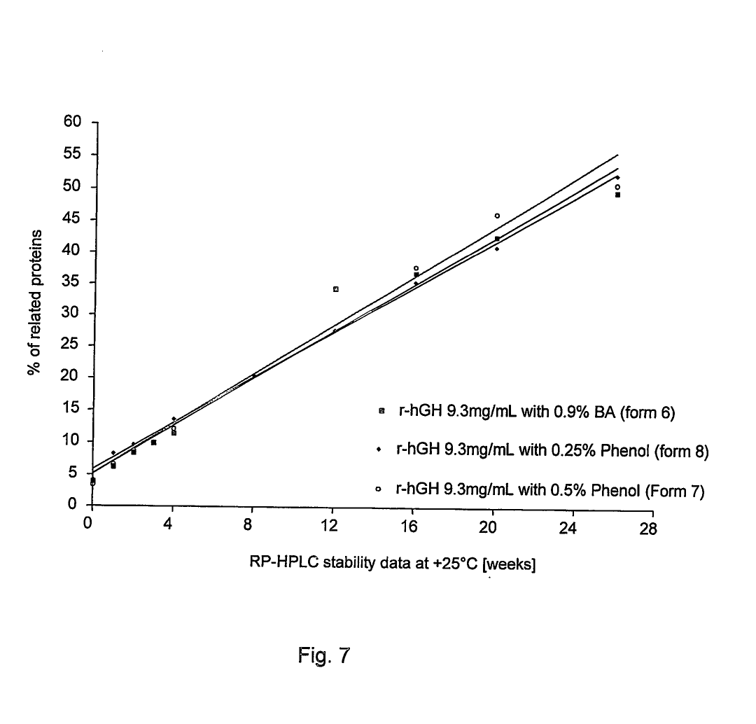

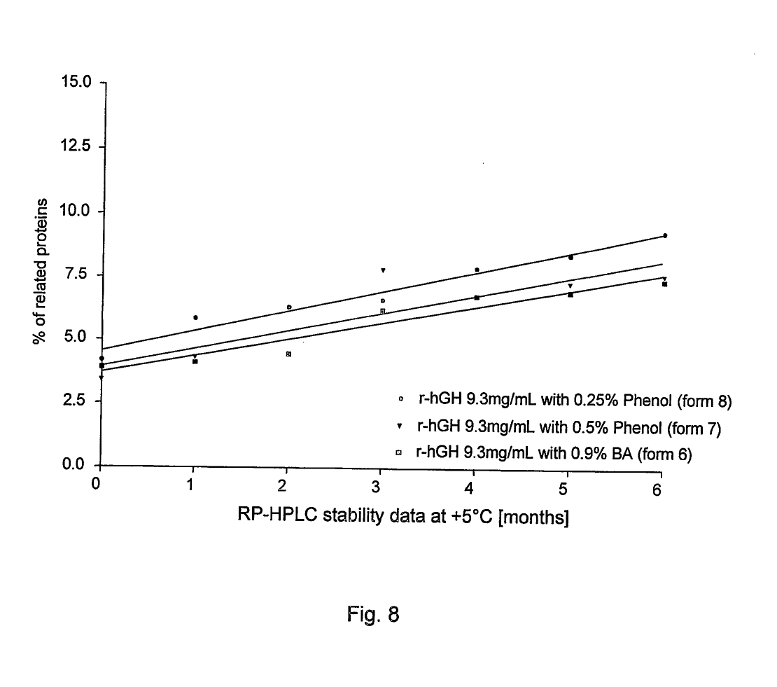

The stability data of these three formulations at +25° C. and +5° C., as measured by percentage of degraded proteins by RP-HPLC, are depicted in FIGS. 7 and 8, respectively.

The following conclusions can be drawn from this experiment:
0.5% Phenol formulation presented abundant particulate matter with consequent decrease of protein content;
No protein content decrease over the stability at +5° C. for 0.9% BA and 0.25% Phenol formulations;
Significant increase of pH during the storage at +25° C. and +5° C. (from 6.0 to 6.4-6.5);
No increase of High Molecular Weight Substances;
No particulate matter in 0.9% BA and 0.25% Phenol solution.

Formulations 9 o 17 (For Multi-Dose Administration):

TABLE 10

| | 9 | 10 | 11 | 12 |
|---|---|---|---|---|
| r-hGH (mg/ml) | 0.8 | 4.0 | 0.8 | 4.0 |
| Sucrose (mg/ml) | 60.0 | 60.0 | 60.0 | 60.0 |
| Benzyl alcohol (mg/ml) | 9.0 | 9.0 | 9.0 | 9.0 |
| Pluronic F 68 (mg/ml) | 1.5 | 1.5 | 1.5 | 1.5 |
| Acetate buffer pH 5.9 | 10 mM | 10 mM | — | — |
| Citric acid | — | — | to pH 5.9 | to pH 5.9 |

TABLE 11

| | 13 | 14 | 15 | 16 | 17 |
|---|---|---|---|---|---|
| r-hGH (mg/ml) | 0.8 | 4.0 | 0.8 | 4.0 | 0.8 |
| Sucrose (mg/ml) | 60.0 | 60.0 | 60.0 | 60.0 | 60.0 |
| Phenol (mg/ml) | 3.0 | 3.0 | 3.0 | 3.0 | 3.0 |
| Pluronic F 68 (mg/ml) | 1.5 | 1.5 | 1.5 | 1.5 | 1.5 |
| Acetate buffer pH 5.9 | 10 mM | 10 mM | — | — | — |
| Citrate buffer pH 5.9 | — | — | — | — | 10 mM |
| Citric acid | — | — | to pH | to pH | — |
| | | | 5.9 | 5.9 | |

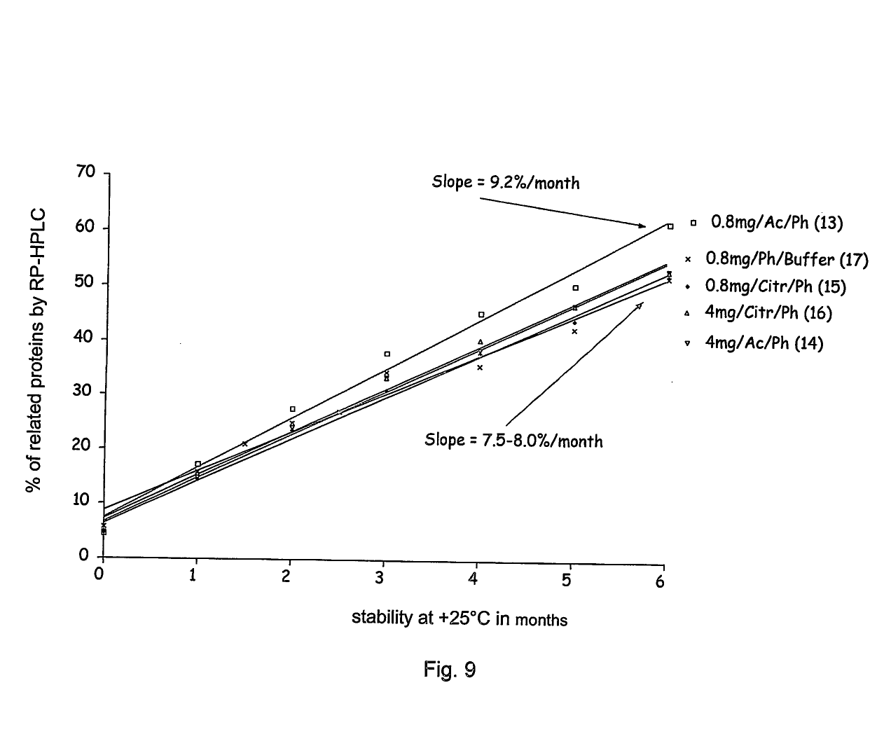

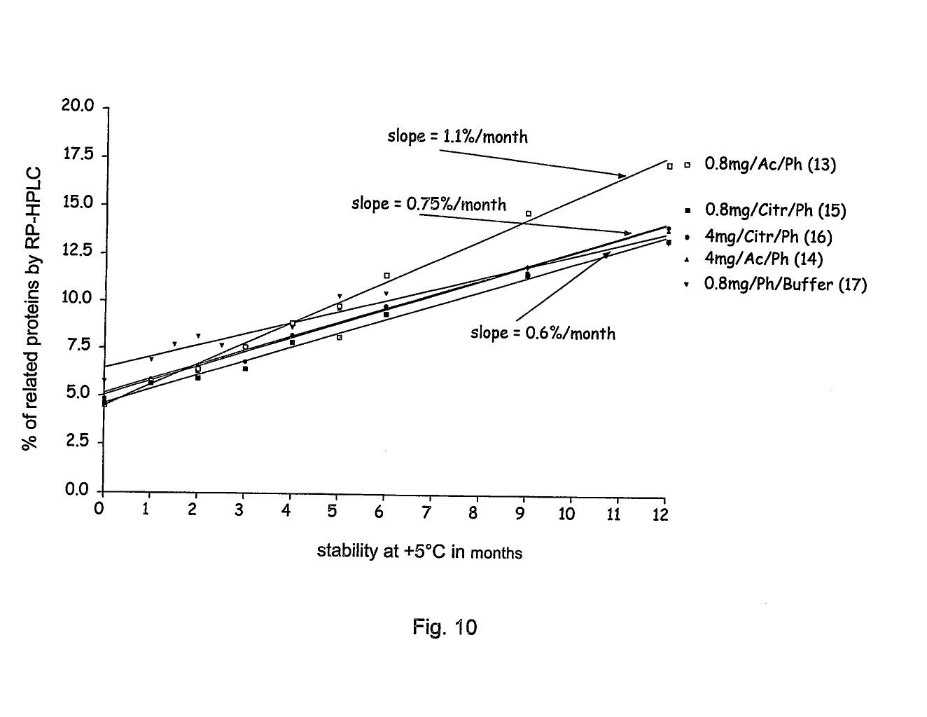

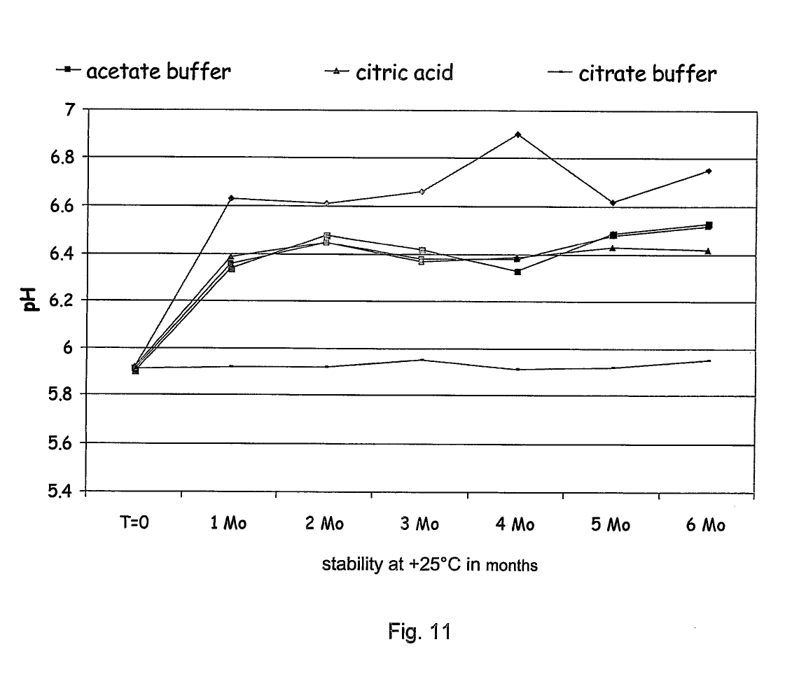

The stability data of these three formulations at +25° C. and +5° C., as measured by percentage of degraded proteins by RP-HPLC, are depicted in FIGS. 9 and 10, respectively. The pH variation at +25° C. over a period of 6 months is depicted in FIG. 11.

The following conclusions can be drawn from this series of experiments:
No protein content decrease over the stability at +5° C.
Significant variation of pH over the stability at +5° C. and +25° C. for acetate and citric acid formulations—not significant for citrate buffer formulation
No increase of High Molecular Weight Substances over the stability at +5° C.
No particulate matter in solution

### REFERENCES

1. Altschul S F et al, J Mol Biol, 215,403-410, 1990
2. Altschul S F et al., Nucleic Acids Res., 25:389-3402, 1997
3. Ausubel et al, Current Protocols in Molecular Biology, Greene Publications and Wiley Interscience (New York, 1987-1998)
4. Becker et al, Biotechnol. Appl. Biochem. 10:326 (1988)
5. Bewly et al, Int. J. Peptide and Protein Res. 4:281-287 (1972)
6. Chen et al, Genomics 4:479-497 (1989)
7. Devereux J et al, Nucleic Acids Res, 12, 387-395, 1984.
8. Gertler et al, Endocrinology 118:720 (1986)
9. Goeddel et al Nature, 281:544 (1979)
10. Graff et al, J. Biol. Chem. 257:2365 (1982)
11. Grantham, Science, Vol. 185, pp. 862-864 (1974).
12. Hsiung et al, Biotechnology 7:267 (1989)
13. Jorgensson et al, Pharmacol. Toxicol. 63:129 (1988)
14. Lewis et al, Endocrinology 101:1587 (1977)
15. Lewis et al, J. Biol. Chem. 253:2679 (1978)
16. Lewis et al, Endocrinology 104:1256 (1979)
17. Lewis et al, Biochem. Biophys. Res. Comm. 92:511 (1980)
18. Lewis et al, J. Biol. Chem. 256:11645 (1981)
19. Martial et al, Science 205:602-607 (1979)
20. Meinkoth J, Wahl G. Hybridization of nucleic acids immobilized on solid supports. Anal Biochem. 1984 May 1;138(2):267-84.
21. Moore et al, Endocrinology 122:2920 (1988)
22. Pearson W R, Methods in Enzymology, 183, 63-99, 1990
23. Pearson W R and Lipman D J, Proc Nat Acad Sci USA, 85, 2444-2448, 1988
24. Pearlman and Nguyen (1989), In D. Marshak and D. Liu (eds), Therapeutic Peptides and Proteins, Formulations, Delivery and Targeting, Current Communications in Molecular Biology, Cold-Spring Harbour Laboratory, Cold Spring Harbour, New York, pp 23-30
25. Singh et al, Endocrinology 94:883 (1974)
26. Smith et al, Science 260:1640-1643 (1998)
27. Smith and Waterman J Mol Biol, 147,195-197, 1981, Advances in Applied Mathematics, 2, 482-489, 1981
28. Thorlacius-Ussing, Neuroendocrinology 43:233 (1987)
29. WO 93/19776
30. WO 94/101398
31. EP-0131864
32. EP-0211601
33. WO 97/29767
34. U.S. Pat. No. 5,567,677

1. A liquid formulation comprising
a) a growth hormone, or a substance, which stimulates release or potentiates the activity of endogenous hGH;
b) polyethylene-polypropylene glycol;
c) a citrate buffer; and
d) a stabilizer.

2. The formulation according to claim 1, wherein the growth hormone is human growth hormone.

3. The formulation according to claim 1, wherein the substance, which stimulates release or potentiates the activity of endogenous hGH, is growth hormone releasing hormone (GHRH).

4. The formulation according to any of the preceding claims claim 1, wherein the stabilizer is sucrose.

5. The formulation according to claim 4, comprising sucrose in a concentration ranging from 10 mg/ml to 100 mg/ml.

6. The formulation according to claim 1, comprising citrate in a concentration ranging from 1 to 100 mM.

7. The formulation according to claim 1, having a pH in the range of 5 to 7.

8. The formulation according to claim 1, comprising the polyethylene-polypropylene glycol in a concentration ranging from 0.5 to 5 mg/ml.

9. The formulation according to claim 1, wherein the polyethylene-polypropylene glycol is a pluronic polyol.

10. The formulation according to claim 9, wherein the pluronic polyol is pluronic F68.

11. The formulation according to claim 1, further comprising a preservative.

12. A formulation according to claim 11, comprising the preservative in a concentration ranging from 1 to 10 mg/ml.

13. The formulation according to claim 11, wherein the preservative is phenol.

14. The formulation according to claim 1, the formulation having a pH of 5.9 and consisting of r-hGH, sucrose, Poloxamer 188, citric acid and/or citrate buffer and optionally water for injection.

15. The formulation according to claim 1, the formulation having a pH of 5.9 and consisting of r-hGH, sucrose, Poloxamer 188, citric acid, phenol and optionally water for injection.

16. A process for production of a liquid formulation according to claim 1, comprising the step of preparing an aqueous solution of the components of (a) to (d).

17. A process for the production of a liquid formulation according to claim 1, comprising the step of placing a predetermined amount of the formulation into a sterile container.

18. A pharmaceutical composition comprising the formulation according to claim 1.

19. The liquid formulation according to claim 1, wherein the formulation is hermetically closed in a sterile condition within a container suited for storage before use.

20. The formulation according to claim 4, comprising sucrose in a concentration ranging from 20 mg/ml to 80 mg/ml.

21. The formulation according to claim 4, comprising sucrose in a concentration at about 60 mg/ml.

22. The formulation according to claim 1, comprising citrate in a concentration ranging from 5 to 50 mM.

23. The formulation according to claim 1, comprising citrate in a concentration ranging from 10 to 20 mM.

24. The formulation according to claim 1, having a pH in the range of 5.5 to 6.5.

25. The formulation according to claim 1, having a pH at about 6.

26. The formulation according to claim 1, comprising the polyethylene-polypropylene glycol in a concentration ranging from 1 to 2 mg/ml.

27. The formulation according to claim 1, comprising the polyethylene-polypropylene glycol in a concentration at 1.5 mg/ml.

28. A formulation according to claim 11, comprising the preservative in a concentration ranging from 2 to 5 mg/ml.

29. A formulation according to claim 11, comprising the preservative in a concentration at 3 mg/ml.
